SEQUENCE LISTING (15 sequences)

SEQ ID NO: 1 - protein, 131 aa, Artificial Sequence
  Humanized antidoby PRO140 VL
  sig_peptide 1...19: signal peptide at amino acids 1-19
MKLPVRLLVLMFWIPASSSDIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIK

SEQ ID NO: 2 - DNA, 429 bp, Artificial Sequence
  Humanized antibody PRO140 VL
tctagaccaccatgaagttgcctgttaggctgttggtgctgatgttctggattcctgcttccagcagtgatattgtgatgacccaatctccactctccctgcctgtcactcctggagagccagcctccatctcttgcagatctagtcagcgccttctgagcagttatggacatacctatttacattggtacctacagaagccaggccagtctccacagctcctgatctacgaagtttccaaccgattttctggggtcccagacaggttcagtggcagtgggtcagggacagatttcacacttaagatcagtagagtggaggctgaggatgtgggagtttattactgctctcaaagtacacatgttcctctcacgttcggacaggggaccaaggtggaaataaaacgtaagtagtcttctcaactctaga

SEQ ID NO: 3 - protein, 141 aa, Artificial Sequence
  Humanized antibody PRO140 #2 VH
  sig_peptide 1...19: signal peptide at amino acids 1-19
MEWSGVFIFLLSVTAGVHSEVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSS

SEQ ID NO: 4 - DNA, 457 bp, Artificial Sequence
  Humanized antibody PRO#2 VH
acgcgtccaccatggaatggagcggagtctttatctttctcctgtcagtaactgcaggtgtccactccgaggtgcagctggtggagtctggtggaggcttggtaaagcctggaggttcccttagactctcctgtgcagcctctggttacactttcagtaactattggatcggatgggtccgccaggctccaggcaaagggctggagtggattggcgatatctaccctggagggaactacatcaggaacaatgagaagttcaaggacaagaccaccctgtcagcagatacttccaagaacacagcctatctgcaaatgaacagcctgaaaaccgaggacacagccgtgtattactgtggaagcagcttcggtagtaactacgtgttcgcctggtttacttactggggccaagggactctggtcacagtctcctcaggtgagtccttaaaacctctaga

SEQ ID NO: 5 - protein, 141 aa, Artificial Sequence
  Humanized antibody PRO140#1 VH
  sig_peptide 1...19: signal peptide at amino acids 1-19
MEWSGVFIFLLSVTAGVHSQVQLVQSGPDVKKPGTSMKMSCKTSGYTFSNYWIGWVRQAPGQGLEWIGDIYPGGNYIRNNEKFKDKTTLTADTSTSTAYMQLGSLRSEDTAVYYCGSSFGSNYVFAWFTYWGQGTLVTVSS

SEQ ID NO: 6 - DNA, 457 bp, Artificial Sequence
  Humanized antibody PRO#1 VH
tctagaccaccatggaatggagcggggtctttatctttctcctgtcagtaactgcaggtgtccactcccaggtccaactggtgcagtctggacctgatgtgaaaaagcctgggacttcaatgaagatgtcctgcaagacgtctggatacaccttcagtaactattggatcggatgggttaggcaggcgcctggacaaggccttgagtggattggagatatttaccctggagggaactatatcaggaacaatgagaagttcaaggacaagaccacactgacggcagacacatcgaccagcacggcctacatgcaacttggcagcctgagatctgaagacactgccgtctattactgtggaagcagcttcggtagtaactacgtgttcgcctggtttacttactggggccaagggactctggtcacagtctcctcaggtgagtccttaaaacctctaga

SEQ ID NO: 7 - protein, 449 aa, Artificial Sequence
  Humanized antibody heavy chain
EVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 219 aa, Artificial Sequence
  Humanized antibody light chain
DIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 16 aa, Artificial Sequence
  Humanized antibody LCDR1
RSSQRLLSSYGHTYLH

SEQ ID NO: 10 - protein, 7 aa, Artificial Sequence
  Humanized antibody LCDR2
EVSNRFS

SEQ ID NO: 11 - protein, 9 aa, Artificial Sequence
  Humanized antibody LCDR3
SQSTHVPLT

SEQ ID NO: 12 - protein, 5 aa, Artificial Sequence
  Humanized antibody HCDR1
NYWIG

SEQ ID NO: 13 - protein, 17 aa, Artificial Sequence
  Humanized antibody HCDR2
DIYPGGNYIRNNEKFKD

SEQ ID NO: 14 - protein, 13 aa, Artificial Sequence
  Humanized antibody HCDR3
SFGSNYVFAWFTY

SEQ ID NO: 15 - protein, 352 aa, Homo sapiens [sequence omitted]

(19) United States
(12) Patent Application Publication
(10) Pub. No.: US 2021/0214448 A1
(43) Pub. Date: Jul. 15, 2021

(54) CCR5 BINDING AGENT FOR THE TREATMENT OF CCR5 POSITIVE METASTATIC CANCER

(71) Applicant: CytoDyn Inc., Vancouver, WA (US)

(72) Inventor: Scott Kelly, Vancouver, WA (US)

(21) Appl. No.: 17/148,281

(22) Filed: Jan. 13, 2021

Related U.S. Application Data
(60) Provisional application No. 62/960,613, filed on Jan. 13, 2020.
(60) Provisional application No. 62/968,954, filed on Jan. 31, 2020.
(60) Provisional application No. 62/977,023, filed on Feb. 14, 2020.

(51) Int. Cl. C07K 16/28; A61P 35/04; A61K 31/282; A61K 39/395; A61K 31/337; A61K 31/4025; A61K 31/4196; A61K 31/506; A61K 31/499; A61K 31/4545; A61K 31/167

(52) U.S. Cl. CPC C07K 16/2866; A61K 2039/505; A61K 31/282; A61K 39/3955; A61K 31/337; A61K 31/4025; A61K 39/39558; A61K 31/4196; A61K 31/506; A61K 31/499; A61K 31/4545; A61K 31/167; A61K 39/39541; C07K 2317/24; C07K 2317/565; A61P 35/04

(57) ABSTRACT

The present disclosure relates to the use of CCR5 binding agents, such as the leronlimab, in the treatment or prevention of CCR5+ metastatic cancer.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 230042_431_SEQUENCE_LISTING.txt. The text file is 15.3 KB, was created on Jan. 7, 2021, and is being submitted electronically via EFS-Web.

### BACKGROUND

Breast cancer continues to be the most common solid tumor affecting women, and it is the second leading cause of cancer-related death in women. Metastasis is the primary cause of death in patients with breast cancer. Currently no treatments exist that are directed specifically to the metastatic process.

Ten to fifteen percent of breast cancer patients have Triple Negative Breast Cancer (TNBC), which is defined by the lack of estrogen receptor (ER), progesterone receptor (PgR) and human epidermal growth factor receptor-2 (HER-2) expression, which are known targets of endocrine therapies and anti-HER2 agents, respectively. Approximately 70-84% of TNBCs are basal-like; conversely, about 70% of basal-like tumors are TNBCs (Nielson 2004, Prat 2011, Prat 2013).

Patients with TNBC are a clinically highly relevant patient group that is characterized by younger age, unfavorable histopathological features including high histological grade, elevated mitotic count, high rate of p53 mutations and pushing margins of invasion with a shortened overall survival (OS) and disease free survival (DFS) compared to other breast cancer subgroups [Dawood, 2011] [Engstrom, 2013][Malorni, 2012]. For these reasons, TNBC accounts for a disproportionately high percentage of metastases, particularly distant recurrence, and death among patients with breast cancer. Moreover, in younger women TNBC has been described to occur more often with a high risk of recurrence and death, respectively, the latter with a peak incidence of 3 years after primary diagnosis. The pattern of recurrence more often involves visceral organs and less common bones compared to other breast cancer subtypes [Foulkes, 2010].

Compared with the hormone receptor-positive breast cancers, TNBC has a worse prognosis, with an aggressive natural history [Lebert 2018]. At diagnosis, TNBC tumors are more likely to be T2 or T3, to be positive for lymphovascular invasion, and to have already metastasized to lymph nodes [Dent 2007]. Metastatic TNBC (mTNBC) accounts for a disproportionately high percentage of metastases, particularly distant recurrence, and death among patients with breast cancer. Currently, no treatments exist that are directed specifically to the metastatic process.

Chemotherapy is still the main treatment option for TNBC patients, and standard treatment is surgery with adjuvant therapy, such as chemotherapy and radiotherapy. Although TNBC responds to chemotherapeutic agents such as taxanes and anthracyclines better than other subtypes of breast cancer, prognosis still remains poor. As a variation, neoadjuvant chemotherapy is frequently used for triple-negative breast cancers [Hudis 2011]. This allows for a higher rate of breast-conserving surgeries and, from evaluating the response to the chemotherapy, gives important clues about the individual responsiveness of the particular cancer to chemotherapy.

Due to the loss of target receptors such as ER, PGR, and HER-2, patients with TNBC do not benefit from hormonal or trastuzumab-based therapy. Hence, surgery and chemotherapy, individually or in combination, appear to be the only available modalities. To date there are multiple approaches attempting to improve care of triple negative breast cancer patients, including DNA damaging agents like platinum, targeted EGFR and VEGF inhibitors, and, PARP inhibitors; however, none have been as clinically successful as anticipated and more targeted therapies need to be developed and explored [Aysola 2013]. Thus, metastatic TNBC is a complex disease with an unmet need and an unproven treatment regimen in clinics.

### BRIEF DESCRIPTION OF THE SEVERAL VIEWS OF THE DRAWINGS

FIG. 1A, FIG. 1B, FIG. 1C, FIG. 1D, FIG. 1E, and FIG. 1F show maraviroc inhibition of lung metastasis in a mouse model. FIG. 1A shows timecourse images of mouse lung metastasis for a mouse treated with maraviroc. FIG. 1B shows photon flux measurements taking weekly during the timecourse. FIG. 1C shows the presence of pulmonary tumors. FIG. 1D is a plot of the percentage of mice with tumors. FIG. 1E shows histologic staining of the area of the slide covered in tumors. FIG. 1F shows tumor area.

FIG. 2 shows a Kaplan-Meier analysis for node-negative breast cancer, stratified by low CCR5 expression (upper line) and high CCR5 expression (lower line).

FIG. 3A and FIG. 3B show expression of CCR5 on Tregs isolated from the tumor microenvironment in lung, breast, and bladder cancer samples. FIG. 3A shows histograms of FACS analysis, and FIG. 3B shows percentages of populations in the sample.

FIG. 4A, FIG. 4B, and FIG. 4C show immunohistochemical staining for CCR5 in tissue samples from a first subject with triple negative breast cancer.

FIG. 5 shows adverse events reported for Patient D enrolled in the study.

FIG. 6 shows measurements of lesion and nodule sizes (in cm or mm) from Patient A in the single patient emergency use study. Lesions and nodules were measured in the breast and liver; metastases are also qualitatively described.

FIG. 7A and FIG. 7B show protein expression levels of CCR5 (FIG. 7A) and PD-L1 (FIG. 7B) on individual CAMLs from Patient A in the single patient emergency use study. Expression was measured by flow cytometry and reported as Mean Fluorescence Intensity (MFI). CCR5 MFI (“CCR5 INT”) was calculated by subtracting background signal of a negative control sample from the experimental value. CAML size was also measured and reported in μM.

FIG. 8 shows immunohistochemical staining for CCR5 in tissue samples from Patient A in the single patient emergency use study.

FIG. 9 shows the amino acid sequence of the light chain variable region of the humanized version of mouse anti-CCR5 antibody PA14 (SEQ ID NO: 1) and the nucleic acid sequence encoding the same (SEQ ID NO: 2). The CDRs are underlined.

FIG. 10 shows the amino acid sequence of a first heavy chain variable region of a humanized version of mouse anti-CCR5 antibody PA14 (SEQ ID NO: 3), and the nucleic acid sequence encoding the same (SEQ ID NO: 4), in accordance with the invention. This heavy chain variable region is present in the antibody designated herein as PRO 140 #2. The CDRs are underlined.

FIG. 11 shows the amino acid sequence of a second heavy chain variable region of a humanized version of mouse humanized anti-CCR5 antibody PA14 (SEQ ID NO: 5) and the nucleic acid sequence encoding the same (SEQ ID NO: 6) in accordance with the invention. This heavy chain variable region is present in the antibody designated herein as PRO 140 #1. The CDRs are underlined.

### DETAILED DESCRIPTION

Although metastasis is the leading cause of death for patients with breast cancer, currently there are no treatments available that are directed to the metastatic process. Thus, better treatments for metastatic cancer, including metastatic breast cancer are needed. Presented herein are methods for treating a subject for metastatic breast cancer by administering to the subject an effective amount of a CCR5 binding agent, such as leronlimab.

Preclinical and clinical data have suggested that chemokine receptors and its ligands, also referred as chemoattractant or chemotactic cytokines, are involved in the process of cancer cells tropism by specific organs [Moser, 2001][Neagu, 2015][Velasco-Velazquez, 2012]. C—C Chemokine receptor type-5 (CCR5) is selectively reexpressed on the surface of tumor cells during the dedifferentiation and transformation process (velasco-velazquez-2012). Velasco-Velazquez et al. have evaluated an analysis of a combined microarray database comprising 2,254 breast cancer samples and showed that expression of CCL5/CCR5 is higher in basal subtypes (over 58% of samples) of breast cancer compared to luminal subtypes [Velasco-Velazquez, 2012]. CCR5 has been shown to be sufficient to induce in vitro invasiveness and metastasis of breast cancer cells that is blocked by CCR5 inhibitors [velasco-velazquez-2012]. CCR5 inhibitors, such as maraviroc, effectively blocked lung metastases in breast cancer tumor model [see section 4].

CCR5 binding agents, including leronlimab (PRO 140), show a significant reduction in tumor volume in a breast cancer tumor model. Another cancer hallmark that CCR5 presents a potential role is the DNA repair pathways. This cancer characteristic attenuates apoptosis and contributes to chemotherapy resistance and tumor cells immortality. Studies have correlated the altered expression of C—C Chemokine Ligand type-5 (CCL5) with disease progression in patients with breast cancer [Luboshits, 1999][Niwa, 2001][Zhang, 2009].

CCR5 binding agents, such as antagonists maraviroc and vicriviroc, dramatically enhanced cell killing mediated by DNA-damaging chemotherapeutic agents. Single-cell analysis revealed CCR5 governs PI3K/Akt, ribosomal biogenesis, and cell survival signaling [Jiao-2018].

The role of CCR5 blockade of the CCL5-CCR5 pathway in immune control of tumors has recently been shown and provided new horizon to target this deadly disease [de Oliveira, 2017, Del Prete, 2017, Lanitis, 2017]. CCR5 immunohistochemistry of biopsies allows to selectively choosing patients with CCR5 expression not only on tumor but on intra-tumor immune cells in the tumor microenvironment.

Targeted therapy with one or more CCR5 binding agents, such as leronlimab (PRO 140), may have a potential to increase overall response rate due to a synergy in DNA crosslink strand break of chemotherapeutic agents, such as carboplatin, and reduce DNA repair secondary to CCR5 binding by leronlimab (PRO 140).

As shown in the Examples presented herein, data from the first patient in the Phase 1b/2 trial showed the patient had no detectable circulating tumor cells (CTCs) or putative metastatic tumor cells in the peripheral blood and additional large reductions in CCR5 expression on cancer-associated cells at 11 weeks of treatment with leronlimab. This patient's data also demonstrated tumor shrinkage of >20% after just a few weeks of treatment. Additionally, data from the patient under the emergency IND protocol with HER2+ metastatic, stage 4, MBC showed no sign of new metastatic spots in the liver, lung and brain during the treatment with leronlimab. These data demonstrate remarkable improvements in patients living with metastatic breast cancer, a deadly disease that requires imminent new treatment options.

Prior to setting forth this disclosure in more detail, it may be helpful to an understanding thereof to provide definitions of certain terms to be used herein. Unless defined otherwise, all technical and scientific terms used herein have the same meaning as is commonly understood by one of skill in the art to which this invention belongs. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as dose, are to be understood to include any integer within the recited range, unless otherwise indicated. As used herein, the term “about” means±20% of the indicated range, value, or structure, unless otherwise indicated.

It should be understood that the terms “a” and “an” as used herein refer to “one or more” of the enumerated components. The use of the alternative (e.g., “or”) should be understood to mean either one, both, or any combination thereof of the alternatives.

As used herein, the terms “include,” “have,” and “comprise” are used synonymously, which terms and variants thereof are intended to be construed as non-limiting.

The term “consisting essentially of” limits the scope of a claim to the specified materials or steps, or to those that do not materially affect the basic characteristics of a claimed invention. For example, a protein domain, region, or module (e.g., a binding domain, hinge region, linker module) or a protein (which may have one or more domains, regions, or modules) “consists essentially of” a particular amino acid sequence when the amino acid sequence of a domain, region, or module or protein includes extensions, deletions, mutations, or any combination thereof (e.g., amino acids at the amino- or carboxy-terminus or between domains) that, in combination, contribute to at most 20% (e.g., at most 15%, 10%, 8%, 6%, 5%, 4%, 3%, 2%, or 1%) of the length of a domain, region, or module or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

A “therapeutically effective amount” or “effective amount” of an antibody, antigen-binding fragment, or composition of this disclosure refers to an amount of the composition sufficient to result in a therapeutic effect, including improved clinical outcome; slowing tumor growth, reducing tumor volume, preventing tumor formation, preventing tumor metastases; reducing the number of circulating tumor cells, epithelial mesenchymal transition cells, and/or cancer associated macrophage-like cells; lessening or alleviating of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously.

As used herein, “stable” or “stable disease” refers to disease that fails to meet criteria for progressive disease nor partial response. As used herein, “progressive disease” refers to at least a 20% increase in the sum of diameters of up to 5 target lesions (2 lesions/organ), taking as reference the smallest sum on study and an absolute lesion increase of at least 5 mm or the appearance of new lesions. A complete response is the disappearance of all target lesions, and a partial response (PR) is defined as at least a 30% decrease in the sum of the target lesions. Stable disease is defined as fitting the criteria neither for progressive disease nor a partial response.

As used herein, “chemokine” means a cytokine that can stimulate leukocyte movement. Chemokines may be characterized as either cys-cys or cys-X-cys depending on whether the two amino terminal cysteine residues are immediately adjacent or separated by one amino acid. It includes but is not limited to CCL5 (also known as RANTES), MIP-la, or SDF-1, or another chemokine which has similar activity.

As used herein, “chemokine receptor” means a member of a homologous family of seven-transmembrane spanning cell surface proteins that bind chemokines.

As used herein, “C—C chemokine receptor 5,” also known as “CCR5” or “CD195” refers to a G protein-coupled receptor expressed on lymphocytes (e.g., NK cells, B cells, T cells), macrophages, dendritic cells, eosinophils, and microglia, which functions as a chemokine receptor for the C—C chemokine group. CCR5's cognate ligands include CCL3, CCL4, CCL3L1, and CCL5. In some embodiments, CCR5 refers to human CCR5. In some embodiments, CCR5 refers to a protein having an amino acid sequence provided in NCBI Reference Sequence: NP_000570.1 (SEQ ID NO:15).

As used herein, “antibody” means an immunoglobulin molecule comprising two heavy chains and two light chains and that recognizes an antigen. The immunoglobulin molecule may derive from any of the commonly known classes or isotypes, including but not limited to IgA, secretory IgA, IgG, and IgM. IgG subclasses are also well known to those in the art and include but are not limited to human IgG1, IgG2, IgG3, and IgG4. It includes, by way of example, both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. Optionally, an antibody can be labeled with a detectable marker. Detectable markers include, for example, radioactive or fluorescent markers. The antibody may be a human or nonhuman antibody. The nonhuman antibody may be humanized by recombinant methods to reduce its immunogenicity in humans. Methods for humanizing antibodies are known to those skilled in the art.

As used herein, “monoclonal antibody,” also designated as “mAb,” is used to describe antibody molecules whose primary sequences are essentially identical and which exhibit the same antigenic specificity. Monoclonal antibodies may be produced by hybridoma, recombinant, transgenic, or other techniques known to one skilled in the art.

As used herein, “heavy chain” means the larger polypeptide of an antibody molecule composed of one variable domain (VH) and three or four constant domains (CHL CH2, CH3, and CH4), or fragments thereof.

As used herein, “light chain” means the smaller polypeptide of an antibody molecule composed of one variable domain (VL) and one constant domain (CL), or fragments thereof.

As used herein, a “binding fragment” or an “antigen-binding fragment or portion” of an antibody refers to the fragment or portion of an intact antibody that has or retains the ability to bind to the antigen target molecule recognized by the intact antibody, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, tetrabodies, tandem di-scFv, and tandem tri-scFv.

As used herein, “Fab” means a monovalent antigen binding fragment of an immunoglobulin that consists of one light chain and part of a heavy chain. It can be obtained by brief papain digestion or by recombinant methods.

As used herein, “F(ab′)2 fragment” means a bivalent antigen binding fragment of an immunoglobulin that consists of both light chains and part of both heavy chains. It can be obtained by brief pepsin digestion or recombinant methods.

As used herein, “CDR” or “complementarity determining region” means a highly variable sequence of amino acids in the variable domain of an antibody.

As used herein, “humanized” describes antibodies wherein some, most or all of the amino acids outside the CDR regions are replaced with corresponding amino acids derived from human immunoglobulin molecules. In one embodiment of the humanized forms of the antibodies, some, most, or all of the amino acids outside the CDR regions have been replaced with amino acids from human immunoglobulin molecules but where some, most, or all amino acids within one or more CDR regions are unchanged. Small additions, deletions, insertions, substitutions, or modifications of amino acids are permissible as long as they would not abrogate the ability of the antibody to bind a given antigen. Suitable human immunoglobulin molecules would include IgG1, IgG2, IgG3, IgG4, IgA, and IgM molecules. A “humanized” antibody would retain a similar antigenic specificity as the original antibody, e.g., in the present disclosure, the ability to bind CCR5.

One skilled in the art would know how to make the humanized antibodies of the subject invention. Various publications, several of which are hereby incorporated by reference into this application, also describe how to make humanized antibodies. For example, the methods described in U.S. Pat. No. 4,816,567 comprise the production of chimeric antibodies having a variable region of one antibody and a constant region of another antibody. U.S. Pat. No. 5,225,539 describes another approach for the production of a humanized antibody. This patent describes the use of recombinant DNA technology to produce a humanized antibody wherein the CDRs of a variable region of one immunoglobulin are replaced with the CDRs from an immunoglobulin with a different specificity such that the humanized antibody would recognize the desired target but would not be recognized in a significant way by the human subject's immune system. Specifically, site directed mutagenesis is used to graft the CDRs onto the framework.

Other approaches for humanizing an antibody are described in U.S. Pat. Nos. 5,585,089 and 5,693,761 and WO 90/07861, which describe methods for producing humanized immunoglobulins. These have one or more CDRs and possible additional amino acids from a donor immunoglobulin and a framework region from an accepting human immunoglobulin. These patents describe a method to increase the affinity of an antibody for the desired antigen. Some amino acids in the framework are chosen to be the same as the amino acids at those positions in the donor rather than in the acceptor. Specifically, these patents describe the preparation of a humanized antibody that binds to a receptor by combining the CDRs of a mouse monoclonal antibody with human immunoglobulin framework and constant regions. Human framework regions can be chosen to maximize homology with the mouse sequence. A computer model can be used to identify amino acids in the framework region which are likely to interact with the CDRs or the specific antigen and then mouse amino acids can be used at these positions to create the humanized antibody.

The above U.S. Pat. Nos. 5,585,089 and 5,693,761 and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid residue at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. The affinity and/or specificity of the binding of the humanized antibody may be increased using methods of directed evolution as described in Wu et al., J. Mol. Biol., 284:151 (1999) and U.S. Pat. Nos. 6,165,793; 6,365,408; and 6,413,774.

The variable regions of the humanized antibody may be linked to at least a portion of an immunoglobulin constant region of a human immunoglobulin. In one embodiment, the humanized antibody contains both light chain and heavy chain constant regions. The heavy chain constant region usually includes CH1, hinge, CH2, CH3, and, sometimes, CH4 region. In one embodiment, the constant regions of the humanized antibody are of the human IgG4 isotype. The antibodies, or binding fragments, disclosed herein may either be labeled or unlabeled. Unlabeled antibodies can be used in combination with other labeled antibodies (second antibodies) that are reactive with a humanized antibody, such as antibodies specific for human immunoglobulin constant regions. Alternatively, the antibodies can be directly labeled. A wide variety of labels can be employed, such as radionuclides, fluors, enzymes, enzyme substrates, enzyme cofactors, enzyme inhibitors, ligands (particularly haptens), etc. Numerous types of immunoassays are available and are well known to those skilled in the art for detection of CCR5-expressing cells or detection of CCR5 modulation on cells capable of expressing CCR5.

In some embodiments, the present disclosure provides use of an anti-CCR5 antibody or antigen binding fragment thereof having a light chain variable region (VL) that is at least 70% identical to SEQ ID NO: 1, at least 75% identical to SEQ ID NO: 1, at least 80% identical to SEQ ID NO: 1, at least 85% identical to SEQ ID NO: 1, at least 90% identical to, or at least 95% identical to SEQ ID NO: 1. In some embodiments, the present disclosure provides use of an anti-CCR5 antibody or antigen binding fragment thereof having a light chain variable antibody region that is 70%-100% identical to SEQ ID NO: 1, 75%-100% identical to SEQ ID NO: 1, 80%-100% identical to SEQ ID NO: 1, 85%-100% identical to SEQ ID NO: 1, 90%-100% identical to SEQ ID NO: for 91%-100% identical to SEQ ID NO: 1.

In some embodiments, the present disclosure provides use of an anti-CCR5 antibody or antigen binding fragment thereof having a light chain variable region (VL) that is at least 70% identical to amino acids 20-131 of SEQ ID NO: 1, at least 75% identical to amino acids 20-131 of SEQ ID NO: 1, at least 80% identical to amino acids 20-131 of SEQ ID NO: 1, at least 85% identical to amino acids 20-131 of SEQ ID NO: 1, at least 90% identical to amino acids 20-131 of SEQ ID NO: 1, or at least 95% identical to amino acids 20-131 of SEQ ID NO: 1. In some embodiments, the present disclosure provides use of an anti-CCR5 antibody or antigen binding fragment thereof having a light chain variable antibody region that is 70%-100% identical to amino acids 20-131 of SEQ ID NO: 1, 75%-100% identical to amino acids 20-131 of SEQ ID NO: 1, 80%-100% identical to amino acids 20-131 of SEQ ID NO: 1, 85%-100% identical to amino acids 20-131 of SEQ ID NO: 1, 90%-100% identical to amino acids 20-131 of SEQ ID NO: for 91%-100% identical to amino acids 20-131 of SEQ ID NO: 1.

In some embodiments, the present disclosure provides use of an anti-CCR5 antibody or antigen binding fragment thereof having a heavy chain variable region (VH) that is at least 70% identical to SEQ ID NO:3, at least 75% identical to SEQ ID NO:3, at least 80% identical to SEQ ID NO:3, at least 85% identical to SEQ ID NO:3, at least 90% identical to SEQ ID NO:3, or at least 95% identical to SEQ ID NO:3. In some embodiments the present disclosure provides use of an anti-CCR5 antibody or antigen binding fragment thereof having a heavy chain antibody variable region that is 70%-100% identical to SEQ ID NO: 3, 75%-100% identical to SEQ ID NO: 3, 80%-100% identical to SEQ ID NO: 3, 85%-100% identical to SEQ ID NO: 3, 90%-100% identical to SEQ ID NO: 3, or 91%-100% identical to SEQ ID NO:3.

In some embodiments, the present disclosure provides use of an anti-CCR5 antibody or antigen binding fragment thereof having a heavy chain variable region (VH) that is at least 70% identical to amino acids 20-141 of SEQ ID NO:3, at least 75% identical to amino acids 20-141 of SEQ ID NO:3, at least 80% identical to amino acids 20-141 of SEQ ID NO:3, at least 85% identical to amino acids 20-141 of SEQ ID NO:3, at least 90% identical to amino acids 20-141 of SEQ ID NO:3, or at least 95% identical to amino acids 20-141 of SEQ ID NO:3. In some embodiments the present disclosure provides use of an anti-CCR5 antibody or antigen binding fragment thereof having a heavy chain antibody variable region that is 70%-100% identical to amino acids 20-141 of SEQ ID NO: 3, 75%-100% identical to amino acids 20-141 of SEQ ID NO: 3, 80%-100% identical to amino acids 20-141 of SEQ ID NO: 3, 85%-100% identical to amino acids 20-141 of SEQ ID NO: 3, 90%-100% identical to amino acids 20-141 of SEQ ID NO: 3, or 91%-100% identical to amino acids 20-141 of SEQ ID NO:3.

In some embodiments, the present disclosure provides use of an anti-CCR5 antibody having a heavy chain variable region (VH) that is at least 70% identical to SEQ ID NO:5, at least 75% identical to SEQ ID NO: 5, at least 80% identical to SEQ ID NO: 5, at least 85% identical to SEQ ID NO: 5, at least 90% identical to SEQ ID NO: 5, or at least 95% identical to SEQ ID NO: 5. In some embodiments the present disclosure provides use of an anti-CCR5 antibody having a heavy chain variable antibody region that is 70%-100% identical to SEQ ID NO: 5, 75%-100% identical to SEQ ID NO: 5, 80%-100% identical to SEQ ID NO: 5, 85%-100% identical to SEQ ID NO: 5, 90%-100% identical to SEQ ID NO: 5, or 91%-100% identical to SEQ ID NO: 5.

In some embodiments, the present disclosure provides use of an anti-CCR5 antibody having a heavy chain variable region (VH) that is at least 70% identical to amino acids 20-141 of SEQ ID NO:5, at least 75% identical to amino acids 20-141 of SEQ ID NO: 5, at least 80% identical to amino acids 20-141 of SEQ ID NO: 5, at least 85% identical to amino acids 20-141 of SEQ ID NO: 5, at least 90% identical to amino acids 20-141 of SEQ ID NO: 5, or at least 95% identical to amino acids 20-141 of SEQ ID NO: 5. In some embodiments the present disclosure provides use of an anti-CCR5 antibody having a heavy chain variable antibody region that is 70%-100% identical to amino acids 20-141 of SEQ ID NO: 5, 75%-100% identical to amino acids 20-141 of SEQ ID NO: 5, 80%-100% identical to amino acids 20-141 of SEQ ID NO: 5, 85%-100% identical to amino acids 20-141 of SEQ ID NO: 5, 90%-100% identical to amino acids 20-141 of SEQ ID NO: 5, or 91%-100% identical to amino acids 20-141 of SEQ ID NO: 5.

In some embodiments, the present disclosure provides use of an anti-CCR5 antibody or an antigen-binding fragment thereof comprising a heavy chain variable region (VH) and a light chain variable region (VL), wherein the VH comprises a heavy chain CDR1 (VH-CDR1) comprising the amino acid sequence of SEQ ID NO:12, a heavy chain CDR2 (VH-CDR2) comprising the amino acid sequence of SEQ ID NO:13, and a heavy chain CDR3 (VH-CDR3) comprising the amino acid sequence of SEQ ID NO:14; and the VL comprises a light chain CDR1 (VL-CDR1) comprising the amino acid sequence of SEQ ID NO:9, a light chain CDR2 (VL-CDR2) comprising the amino acid sequence of SEQ ID NO:10, and a light chain CDR3 (VL-CDR3) comprising the amino acid sequence of SEQ ID NO:11. In some such embodiments, the VH comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO:3 or amino acids 20-141 of SEQ ID NO:3, and a VL comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO:1 or amino acids 20-131 of SEQ ID NO:1, provided that the amino acid sequences of the VH-CDRs (SEQ ID NOS:12-14) and VL-CDRs (SEQ ID NOS:9-11) are unchanged; or the VH comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO:5 or amino acids 20-141 of SEQ ID NO:5, and a VL comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO:1 or amino acids 20-131 of SEQ ID NO:1, provided that the amino acid sequences of the VH-CDRs (SEQ ID NOS:12-14) and VL-CDRs (SEQ ID NOS:9-11) are unchanged.

In some embodiments, the present disclosure provides use of an anti-CCR5 antibody or an antigen-binding fragment thereof comprising: (a) a VH comprising an amino acid sequence of SEQ ID NO:3 or amino acids 20-141 of SEQ ID NO:3, and a VL comprising an amino acid sequence of SEQ ID NO:1 or amino acids 20-131 of SEQ ID NO:1; or (b) a VH comprising an amino acid sequence of SEQ ID NO:5 or amino acids 20-141 of SEQ ID NO:5, and a VL comprising an amino acid sequence of SEQ ID NO:1 or amino acids 20-131 of SEQ ID NO:1.

In some embodiments, the present disclosure provides use of an anti-CCR5 antibody that comprises a heavy chain (HC) and a light chain (LC), wherein the HC comprises an amino acid sequence that has at least 90%, 91%, 92%, 93%, 94%, 95%, 96%, 97%, 98%, 99%, or 100% identity with the amino acid sequence of SEQ ID NO:7, and the LC comprises an amino acid sequence that has at least 90%, 91%, 92%, 93%, 94%, 95%, 96%, 97%, 98%, or 99% identity with the amino acid sequence of SEQ ID NO:8

In some embodiments, the present disclosure provides use of an anti-CCR5 antibody that comprises a HC comprising an amino acid sequence that has the amino acid sequence of SEQ ID NO:7, and a LC comprising an amino acid sequence that has the amino acid sequence of SEQ ID NO:8.

The present disclosure also provides antibody or antibody fragment-polymer conjugates having an effective size or molecular weight that confers an increase in serum half-life, an increase in mean residence time in circulation (MRT), and/or a decrease in serum clearance rate over underivatized antibody fragments. Antibody fragment-polymer conjugates can be made by derivatizing the desired antibody fragment with an inert polymer. It will be appreciated that any inert polymer which provides the conjugate with the desired apparent size or which has the selected actual molecular weight is suitable for use in constructing antibody fragment-polymer conjugates of the invention.

Many inert polymers are suitable for use in pharmaceuticals. See, e.g., Davis et al., Biomedical Polymers: Polymeric Materials and Pharmaceuticals for Biomedical Use, pp. 441-451 (1980). For the antibody or antibody fragment-polymer conjugates disclosed herein, a non-proteinaceous polymer is used. The nonproteinaceous polymer ordinarily is a hydrophilic synthetic polymer, i.e., a polymer not otherwise found in nature. However, polymers which exist in nature and are produced by recombinant or in vitro methods are also useful, as are polymers which are isolated from native sources. Hydrophilic polyvinyl polymers fall within the scope of this invention, e.g., polyvinyl alcohol and polyvinylpyrrolidone. Particularly useful are polyalkylene ethers such as polyethylene glycol (PEG); polyoxyalklyenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g., polymannuronic acid, or alginic acid), D-glucosamine, D-galactosamine, D-glucose, and neuraminic acid including homopolysaccharides and heteropolysaccharides such as lactose, amylopectin, starch, hydroxyethyl starch, amylose, dextran sulfate, dextran, dextrins, glycogen, or the polysaccharide subunit of acid mucopolysaccharides, e.g., hyaluronic acid, polymers of sugar alcohols such as polysorbitol and polymannitol, heparin, or heparon. The polymer prior to cross-linking need not be, but preferably is, water soluble but the final conjugate must be water soluble. Preferably, the conjugate exhibits a water solubility of at least about 0.01 mg/ml and more preferably at least about 0.1 mg/ml, and still more preferably at least about 1 mg/ml. In one embodiment, the polymer should not be highly immunogenic in the conjugate form, nor should it possess viscosity that is incompatible with intraveneous infusion or injection if the conjugate is intended to be administered by such routes.

In one embodiment, the polymer contains only a single group which is reactive. This helps to avoid cross-linking of protein molecules. However it is within the scope of the invention to maximize reaction conditions to reduce cross-linking, or to purify the reaction products through gel filtration or ion-exchange chromatography to recover substantially homogeneous derivatives. In other embodiments, the polymer contains two or more reactive groups for the purpose of linking multiple antibody fragments to the polymer backbone.

Gel filtration or ion-exchange chromatography can be used to recover the desired derivative in substantially homogeneous form.

The molecular weight of the polymer can range up to about 500,000 D and preferably is at least about 20,000 D, or at least about 30,000 D, or at least about 40,000 D. The molecular weight chosen can depend upon the effective size of the conjugate to be achieved, the nature (e.g., structure such as linear or branched) of the polymer and the degree of derivitization, i.e., the number of polymer molecules per antibody fragment, and the polymer attachment site or sites on the antibody fragment.

The polymer can be covalently linked to the antibody fragment through a multifunctional crosslinking agent which reacts with the polymer and one or more amino acid residues of the antibody fragment to be linked. However, it is also within the scope of the invention to directly crosslink the polymer by reacting a derivatized polymer with the antibody fragment, or vice versa.

The covalent crosslinking site on the antibody fragment includes the N-terminal amino group and epsilon amino groups found on lysine residues, as well other amino, imino, carboxyl, sulfhydryl, hydroxyl, or other hydrophilic groups. The polymer may be covalently bonded directly to the antibody fragment without the use of a multifunctional (ordinarily bifunctional) crosslinking agent, as described in U.S. Pat. No. 6,458,355.

The degree of substitution with such a polymer will vary depending upon the number of reactive sites on the antibody fragment, the molecular weight, hydrophilicity and other characteristics of the polymer, and the particular antibody fragment derivitization sites chosen. In general, the conjugate contains from 1 to about 10 polymer molecules, but greater numbers of polymer molecules attached to the antibody fragments of the invention are also contemplated. The desired amount of derivitization is easily achieved by using an experimental matrix in which the time, temperature, and other reaction conditions are varied to change the degree of substitution, after which the level of polymer substitution of the conjugates is determined by size exclusion chromatography or other means known in the art. Functionalized PEG polymers to modify the antibody fragments of the invention are available from Shearwater Polymers, Inc. (Huntsville, Ala.). Such commercially available PEG derivatives include, but are not limited to, amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-oxycarbonylimidazole, PEG-nitrophenyl carbonate, PEG tresylate, PEG-glycidyl ether, PEG-aldehyde, PEG-vinylsulfone, PEG-maleimide, PEG-orthopyridyl-disulfide, heterofunctional PEGs, PEG vinyl derivatives, PEG silanes, and PEG phospholides. The reaction conditions for coupling these PEG derivatives will vary depending on the protein, the desired degree of PEGylation, and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc. Specific instructions for the use of any particular derivative are available from the manufacturer. The conjugates of which may be separated from the unreacted starting materials by gel filtration or ion exchange HPLC.

As used herein, “anti-chemokine receptor antibody” means an antibody which recognizes and binds to an epitope on a chemokine receptor. As used herein, “anti-CCR5 antibody” means a monoclonal antibody that recognizes and binds to an epitope on the CCR5 chemokine receptor.

As used herein, “epitope” means a portion of a molecule or molecules that forms a surface for binding antibodies or other compounds. The epitope may comprise contiguous or noncontiguous amino acids, carbohydrate, or other nonpeptidyl moieties or oligomer-specific surfaces.

As used herein, “polypeptide” means two or more amino acids linked by a peptide bond.

A “nucleic acid molecule,” or “polynucleotide,” may be in the form of RNA or DNA, which includes cDNA, genomic DNA, and synthetic DNA. A nucleic acid molecule may be double stranded or single stranded, and if single stranded, may be the coding strand or non-coding (anti-sense strand). A coding molecule may have a coding sequence identical to a coding sequence known in the art or may have a different coding sequence, which, as the result of the redundancy or degeneracy of the genetic code, or by splicing, can encode the same polypeptide.

“Analogs” of antibodies or binding fragments include molecules differing from the antibodies or binding fragments by conservative amino acid substitutions. For purposes of classifying amino acid substitutions as conservative or non-conservative, amino acids may be grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Due to the degeneracy of the genetic code, a variety of nucleic acid sequences encode the proteins or polypeptides disclosed herein. For example, homologous nucleic acid molecules may comprise a nucleotide sequence that is at least about 90% identical to a reference nucleotide sequence. More preferably, the nucleotide sequence is at least about 95% identical, at least about 97% identical, at least about 98% identical, or at least about 99% identical to a reference nucleotide sequence. The homology can be calculated using various, publicly available software tools well known to one of ordinary skill in the art. Exemplary tools include the BLAST system available from the website of the National Center for Biotechnology Information (NCBI) at the National Institutes of Health.

One method of identifying highly homologous nucleotide sequences is via nucleic acid hybridization. Thus, homologous nucleic acid molecules hybridize under high stringency conditions. Identification of related sequences can also be achieved using polymerase chain reaction (PCR) and other amplification techniques suitable for cloning related nucleic acid sequences. Preferably, PCR primers are selected to amplify portions of a nucleic acid sequence of interest, such as a CDR.

The term “high stringency conditions” as used herein refers to parameters with which the art is familiar. Nucleic acid hybridization parameters may be found in references that compile such methods, e.g., Molecular Cloning: A Laboratory Manual, J. Sambrook, et al., eds., Second Edition, Cold Spring Harbor Laboratory Press, Cold Spring Harbor, N.Y., (1989), or Current Protocols in Molecular Biology, F. M. Ausubel, et al., eds., John Wiley & Sons, Inc., New York. One example of high stringency conditions is hybridization at 65 degrees Centigrade in hybridization buffer (3.5×SSC, 0.02% Ficoll, 0.02% polyvinyl pyrrolidone, 0.02% Bovine Serum Albumin, 2.5 mM NaH2PO4 (pII7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, a membrane upon which the nucleic acid is transferred is washed, for example, in 2×SSC at room temperature and then at 0.1-0.5×SSC/0.1×SDS at temperatures up to 68 degrees Centigrade.

CCR5 Binding Agent

In one aspect, the present disclosure relates to the use of CCR5 binding agents for use in methods of treating and/or preventing CCR5 positive metastatic breast cancer.

In one embodiment, the present disclosure provides for the use of leronlimab (also referred to as PRO140), or binding fragment thereof, in treating or preventing CCR5 positive metastatic breast cancer. PRO 140 is a humanized monoclonal antibody described in U.S. Pat. Nos. 7,122,185 and 8,821,877, which are incorporated herein by reference, in their entirety. PRO 140 is a humanized version of the murine mAb, PA14, which was generated against CD4⁺CCR5⁺ cells. Olson et al., Differential Inhibition of Human Immunodeficiency Virus Type 1 Fusion, gp 120 Binding and CC-Chemokine Activity of Monoclonal Antibodies to CCR5, J. Virol., 73: 4145-4155. (1999). PRO 140 binds to CCR5 expressed on the surface of a cell, and potently inhibits HIV-1 entry and replication at concentrations that do not affect CCR5 chemokine receptor activity in vitro and in the hu-PBL-SCID mouse model of HIV-1 infection. Olson et al., Differential Inhibition of Human Immunodeficiency Virus Type 1 Fusion, gp 120 Binding and CC-Chemokine Activity of Monoclonal Antibodies to CCR5, J. Virol., 73: 4145-4155. (1999); Trkola et al., Potent, Broad-Spectrum Inhibition of Human Immunodeficiency Virus Type 1 by the CCR5 Monoclonal Antibody PRO 140, J. Virol., 75: 579-588 (2001).

Nucleic acids encoding heavy and light chains of the humanized PRO 140 antibody have been deposited with the ATCC. Specifically, the plasmids designated pVK-HuPRO140, pVg4-HuPRO140 (mut B+D+I) and pVg4-HuPRO140 HG2, respectively, were deposited pursuant to, and in satisfaction of, the requirements of the Budapest Treaty with the ATCC, Manassas, Va., U.S.A. 20108, on Feb. 22, 2002, under ATCC Accession Nos. PTA 4097, PTA 4099, and PTA 4098, respectively.

In a one embodiment, the methods disclosed herein comprise administering a humanized antibody designated PRO 140 or an antibody that competes with PRO 140 for binding to the CCR5 receptor, wherein the PRO 140 comprises (i) two light chains, each light chain comprising the expression product of the plasmid designated pVK:HuPRO140-VK (ATCC Deposit Designation PTA-4097), and (ii) two heavy chains, each heavy chain comprising the expression product of either the plasmid designated pVg4:HuPRO140 HG2-VH (ATCC Deposit Designation PTA-4098) or the plasmid designated pVg4:HuPRO140 (mut B+D+I)-VH (ATCC Deposit Designation PTA-4099). In a further embodiment, the PRO 140 is a humanized or human antibody that binds to the same epitope as that to which antibody PRO 140 binds. In another embodiment, the monoclonal antibody is the humanized antibody designated PRO 140.

In a further embodiment, the present disclosure relates to the use of the human antibody designated CCR5mAb004, or a binding fragment thereof. CCR5mAb004 is a fully human mAb, generated using the Abgenix XenoMouse® technology, that specifically recognizes and binds to CCR5. See Roschke et al., Characterization of a Panel of Novel Human Monoclonal Antibodies That Specifically Antagonize CCR5 and Block HIV Entry, 44th Annual Interscience Conference on Antimicrobial Agents and Chemotherapy, Washington, D.C., Oct. 30-Nov. 2, 2004 (2004); HGS Press Release, Human Genome Sciences Characterizes Panel of Novel Human Monoclonal Antibodies That Specifically Antagonize the CCR5 Receptor and Block HIV-1 Entry, Nov. 2, 2004 (2004); HGS Press Release, Human Genome Sciences Begins Dosing of Patients in a Phase 1 Clinical Trial of CCR5 mAb in Patients Infected With HIV-1, Mar. 30, 2005 (2005).

In one embodiment, the present disclosure relates to the use of the monoclonal antibody PA14, produced by the hybridoma cell line designated PA14 (ATCC Accession No. HB-12610), a binding fragment thereof, or an antibody that competes with monoclonal antibody PA-14 in binding to the CCR5 receptor, in treating or preventing cancer.

In one embodiment of the methods described herein, the antibody or binding fragment thereof comprises a light chain of the antibody. In another embodiment, the antibody or binding fragment thereof comprises a heavy chain of the antibody. In a further embodiment, the antibody or binding fragment thereof comprises an Fab portion of the antibody. In a still further embodiment, the antibody or binding fragment thereof comprises an F(ab′)2 portion of the antibody. In an additional embodiment, the antibody or binding fragment thereof comprises an Fd portion of the antibody. In another embodiment, the antibody or binding fragment thereof comprises an Fv portion of the antibody. In a further embodiment, the antibody or binding fragment thereof comprises a variable domain of the antibody. In a still further embodiment, the antibody or binding fragment thereof comprises one or more CDR domains of the antibody. In yet another embodiment, the antibody or binding fragment thereof comprises six CDR domains of the antibody.

Methods of Treating Metastatic Breast Cancer and Solid Tumors

In one aspect, the present disclosure provides methods of treating or preventing metastatic breast cancer comprising administering to a subject in need thereof a CCR5 binding agent.

In one embodiment, the present disclosure provides a method of treating or preventing CCR5 positive metastatic breast cancer comprising administering to a subject in need thereof an effective amount of a CCR5 binding agent.

In a further embodiment, the CCR5 binding agent competes with CCL5 for binding to the CCR5 cell receptor. In a further embodiment, the CCR5 binding agent comprises the monoclonal antibody PA14, leronlimab, or CCR5mAb004, or a binding fragment thereof. In a further embodiment, the competitive inhibitor competes for binding with the monoclonal antibody PA14, leronlimab, or CCR5mAb004, or a binding fragment thereof.

In one embodiment, the present disclosure provides a method of treating or preventing CCR5 positive metastatic breast cancer comprising administering to a subject in need thereof leronlimab, or binding fragment thereof.

In one aspect, the present disclosure provides methods of treating or preventing solid tumors comprising administering to a subject in need thereof a CCR5 binding agent.

In one embodiment, the present disclosure provides a method of treating or preventing CCR5 positive solid tumors comprising administering to a subject in need thereof an effective amount of a CCR5 binding agent.

In a further embodiment, the CCR5 binding agent competes with CCL5 for binding to the CCR5 cell receptor. In a further embodiment, the CCR5 binding agent comprises the monoclonal antibody PA14, leronlimab, or CCR5mAb004, or a binding fragment thereof. In a further embodiment, the competitive inhibitor competes for binding with the monoclonal antibody PA14, leronlimab, or CCR5mAb004, or a binding fragment thereof.

In one embodiment, the present disclosure provides a method of treating or preventing CCR5 positive solid tumors comprising administering to a subject in need thereof leronlimab, or binding fragment thereof.

In any of the aforementioned embodiments, preventing the metastatic breast cancer or solid tumor may comprise administering to a subject in need thereof leronlimab, or binding fragment thereof as an adjuvant therapy. The term “adjuvant therapy”, as used herein, refers to additional treatment given after the primary treatment to decrease the chances of disease recurrence. In some instances, adjuvant therapy is administered after surgery where all detectable disease has been removed, but where there remains a statistical risk of relapse due to undetectable disease.

In any of the aforementioned embodiments, preventing the metastatic breast cancer may comprise slowing the growth or spread of the cancer metastasis or the primary tumor, preventing the formation of a metastatic tumor, or limiting or reducing the growth or size of a metastatic tumor or primary tumor.

In any of the aforementioned embodiments, preventing the solid tumor may comprise slowing the growth or spread of the cancer metastasis or the primary tumor, preventing the formation of a metastatic tumor, or limiting or reducing the growth or size of a metastatic tumor or primary tumor.

In one embodiment, CCR5 binding agent, such as leronlimab, is administered with a pharmaceutically acceptable carrier. Pharmaceutically acceptable carriers are well known to those skilled in the art. Such pharmaceutically acceptable carriers may include but are not limited to aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline, and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In one embodiment, the CCR5 binding agent is provided in a formulation as disclosed in U.S. Pat. No. 9,956,165, the contents of which are incorporated here by this reference.

The dose of the composition of the invention will vary depending on the subject and upon the particular route of administration used. Dosages can range from 0.1 to 100,000 μg/kg. Based upon the composition, the dose can be delivered continuously, such as by continuous pump, or at periodic intervals, e.g., on one or more separate occasions. Desired time intervals of multiple doses of a particular composition can be determined without undue experimentation by one skilled in the art.

In one embodiment of the instant methods, the antibody or binding fragment thereof is administered to the subject a plurality of times and each administration delivers from 0.01 mg per kg body weight to 50 mg per kg body weight of the antibody or binding fragment thereof to the subject. In another embodiment, each administration delivers from 0.05 mg per kg body weight to 25 mg per kg body weight of the antibody or binding fragment thereof to the subject. In a further embodiment, each administration delivers from 0.1 mg per kg body weight to 10 mg per kg body weight of the antibody or binding fragment thereof to the subject. In a still further embodiment, each administration delivers from 0.5 mg per kg body weight to 5 mg per kg body weight of the antibody or binding fragment thereof to the subject. In another embodiment, each administration delivers from 1 mg per kg body weight to 3 mg per kg body weight of the antibody or binding fragment thereof to the subject. In another embodiment, each administration delivers about 2 mg per kg body weight of the antibody or binding fragment thereof to the subject. Embodiments include dosages in amounts ranging from about 175 mg to about 1,400 mg, including dosage forms delivering certain amounts of the CCR5 binding agent such as 175 mg, 350 mg, 525 mg, 700 mg, 875 mg, 1050 mg, 1,225 mg, and 1,400 mg.

In one embodiment, the antibody or binding fragment thereof is administered a plurality of times, and a first administration is separated from the subsequent administration by an interval of less than one week. In another embodiment, the first administration is separated from the subsequent administration by an interval of at least one week. In a further embodiment, the first administration is separated from the subsequent administration by an interval of one week. In another embodiment, the first administration is separated from the subsequent administration by an interval of two to four weeks. In another embodiment, the first administration is separated from the subsequent administration by an interval of two weeks. In a further embodiment, the first administration is separated from the subsequent administration by an interval of four weeks. In yet another embodiment, the antibody or binding fragment thereof is administered a plurality of times, and a first administration is separated from the subsequent administration by an interval of at least one month.

In a further embodiment, the antibody or binding fragment thereof is administered to the subject via intravenous infusion. In another embodiment, the antibody or binding fragment thereof is administered to the subject via subcutaneous injection. In another embodiment, the antibody or binding fragment thereof is administered to the subject via intramuscular injection.

In one embodiment, the aforementioned methods may further comprise administering to the subject a cellular therapy, e.g., an autologous or allogeneic immunotherapy; a small molecule; a chemotherapeutic agent; or an inhibitor of CCR5/CCL5 signaling. In one embodiment, an inhibitor of CCR5/CCL5 signaling is administered, and comprises maraviroc, vicriviroc, aplaviroc, SCH-C, TAK-779, PA14 antibody, 2D7 antibody, RoAb13 antibody, RoAb14 antibody, or 45523 antibody.

In one embodiment, the competitive inhibitor to a CCR5 cell receptor, such as PRO 140, is administered in combination with one or more other therapeutic molecules or treatment, such a cellular therapy, e.g., an autologous or allogeneic immunotherapy; a small molecule; a chemotherapeutic; or an inhibitor of CCR5/CCL5 signaling, such as maraviroc, vicriviroc, aplaviroc, SCH-C, TAK-779, PA14 antibody, 2D7 antibody, RoAb13 antibody, RoAb14 antibody, or 45523 antibody. In one embodiment, the methods disclosed herein comprise administering PRO 140 in combination with maraviroc, vicriviroc, aplaviroc, SCH-C, TAK-779, PA14 antibody, 2D7 antibody, RoAb13 antibody, RoAb14 antibody, or 45523 antibody.

In particular embodiments, the methods disclosed herein comprise administering leronlimab in combination with carboplatin. In particular embodiments, the metastatic breast cancer comprises metastatic triple negative breast cancer and the method comprises administering leronlimab in combination with carboplatin.

In particular embodiments, the methods disclosed herein comprise administering leronlimab in combination with herceptin and pertuzumab. In particular embodiments, the metastatic breast cancer comprises HER2+ breast cancer and the method comprises administering leronlimab in combination with herceptin and pertuzumab.

In one embodiment, the CCR5 binding agent, such as PRO 140, is administered in combination with one or more chemotherapeutics such as, for example: alkylating agents such as thiotepa and cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethylenethiophosphaoramide, and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas such as carmustine, chlorozotocin, fotemustine, lomustine, nimustine, and ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogues such as denopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, floxuridine, and 5-FU; androgens such as calusterone, dromostanolone propionate, epitiostanol, mepitiostane, and testolactone; anti-adrenals such as aminoglutethimide, mitotane, and trilostane; folic acid replenishers such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (Taxol™, Bristol-Myers Squibb Oncology, Princeton, N.J.) and docetaxel (Taxotere™, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; and capecitabine; and pharmaceutically acceptable salts, acids, or derivatives of any of the above.

As used herein, a “small-molecule” CCR5 receptor antagonist includes, for example, a small organic molecule which binds to a CCR5 receptor and inhibits the activity of the receptor. In one embodiment, the small molecule has a molecular weight less than 1,500 daltons. In another embodiment, the small molecule has a molecular weight less than 600 daltons.

In one embodiment, the CCR5 binding agent, such as PRO 140, is administered in combination with one or more small molecules, such as SCH-C(Strizki et al., PNAS, 98: 12718-12723 (2001)); SCH-D (SCH 417670; vicriviroc); UK-427,857 (maraviroc; 1-[(4,6-dimethyl-5-pyrimidinyl) carbonyl]-4-[4-[2-methoxy-1(R)-4-(trifluoromethyl)phenyl]ethyl-3(S)-methyl-1-piperazinyli-4-methylpiperidine); GW873140; TAK-652; TAK-779; AMD070; AD101; 1,3,4-trisubstituted pyrrolidines (Kim et al., Bioorg. Med. Chem. Lett., 15: 2129-2134 (2005)); modified 4-piperidinyl-2-phenyl-1-(phenylsulfonylamino)-butanes (Shah et al., Bioorg. Med. Chem. Lett., 15: 977-982 (2005)); Anibamine TFA, Ophiobolin C, or 19,20-epoxycytochalasin Q (Jayasuriya et al., J. Nat. Prod., 67: 1036-1038 (2004)); 5-(piperidin-1-yl)-3-phenyl-pentylsulfones (Shankaran et al., Bioorg. Med. Chem. Lett., 14: 3589-3593 (2004)); 4-(heteroarylpiperdin-1-yl-methyl)-pyrrolidin-1-yl-acetic acid antagonists (Shankaran et al., Bioorg. Med. Chem. Lett., 14: 3419-3424 (2004)); agents containing 4-(pyrazolyl)piperidine side chains (Shu et al., Bioorg. Med. Chem. Lett., 14: 947-52 (2004); Shen et al., Bioorg. Med. Chem. Lett., 14: 935-939 (2004); Shen et al., Bioorg. Med. Chem. Lett., 14: 941-945 (2004)); 3-(pyrrolidin-1-yl)propionic acid analogues (Lynch et al., Org. Lett., 5: 2473-2475 (2003)); [2-(R)-[N-methyl-N-(1-(R)-3-(S)-((4-(3-benzyl-1-ethyl-(1H)-pyrazol-5-yl)piperidin-1-yl)methyl)-4-(S)-(3-fluorophenyl)cyclopent-1-yl)amino]-3-methylbutanoic acid (MRK-1)] (Kumar et al., J. Pharmacol. Exp. Ther., 304: 1161-1171 (2003)); 1,3,4 trisubstituted pyrrolidines bearing 4-aminoheterocycle substituted piperidine side chains (Willoughby et al., Bioorg. Med. Chem. Lett., 13: 427-431 (2003); Lynch et al., Bioorg. Med. Chem. Lett., 12: 3001-3004 (2003); Lynch et al., Bioorg. Med. Chem. Lett., 13: 119-123 (2003); Hale et al., Bioorg. Med. Chem. Lett., 12: 2997-3000 (2002)); bicyclic isoxazolidines (Lynch et al., Bioorg. Med. Chem. Lett., 12: 677-679 (2002)); combinatorial synthesis of CCR5 antagonists (Willoughby et al., Bioorg. Med. Chem. Lett., 11: 3137-41 (2001)); heterocycle-containing compounds (Kim et al., Bioorg. Med. Chem. Lett., 11: 3103-3106 (2001)); antagonists containing hydantoins (Kim et al., Bioorg. Med. Chem. Lett., 11: 3099-3102 (2001)); 1,3,4 trisubstituted pyrrolidines (Hale et al., Bioorg. Med. Chem. Lett., 11: 2741-2745 (2001)); 1-[N-(methyl)-N-(phenylsulfonyl)amino]-2-(phenyl)-4-(4-(N-(alkyl)-N-(benzyloxycarbonyl)amino)piperidin-1-yl)butanes (Finke et al., Bioorg. Med. Chem. Lett., 11: 2475-2479 (2001)); compounds from the plant Lippia alva (Hedge et al., Bioorg. Med. Chem. Lett., 12: 5339-5342 (2004)); piperazine-based CCR5 antagonists (Tagat et al., J. Med. Chem., 47: 2405-2408 (2004)); oximino-piperidino-piperidine-based CCR5 antagonists (Palani et al., Bioorg. Med. Chem. Lett., 13: 709-712 (2003)); rotamers of SCH 351125 (Palani et al., Bioorg. Med. Chem. Lett., 13: 705-708 (2003)); piperazine-based symmetrical heteroaryl carboxamides (McCombie et al., Bioorg. Med. Chem. Lett., 13: 567-571 (2003)); oximino-piperidino-piperidine amides (Palani et al., J. Med. Chem., 45: 3143-3160 (2002)); Sch-351125 and Sch-350634 (Este, Curr. Opin. Investig. Drugs., 3: 379-383 (2002)); 1-[(2,4-dimethyl-3-pyridinyl)carbonyl]-4-methyl-4-[3(S)-methyl-4-[1(S)-[4-(trifluoromethyl)phenyl]ethyl]-1-piperazinyl]-piperidine N1-oxide (Sch-350634) (Tagat et al., J. Med. Chem., 44: 3343-3346 (2001)); 4-[(Z)-(4-bromophenyl)-(ethoxyimino)methyl]-1′-[(2,4-dimethyl-3-pyridinyl)carbonyl]-4′-methyl-1,4′-bipiperidine N-oxide (SCH 351125) (Palani et al., J. Med. Chem., 44: 3339-3342 (2001)); 2(S)-methyl piperazines (Tagat et al., Bioorg. Med. Chem. Lett., 11: 2143-2146 (2001)); piperidine-4-carboxamide derivatives (Imamura et al., Bioorg. Med. Chem., 13: 397-416, 2005); 1-benzazepine derivatives containing a sulfoxide moiety (Seto et al., Bioorg. Med. Chem. Lett., 13: 363-386 (2005)); anilide derivatives containing a pyridine N-oxide moiety (Seto et al., Chem. Pharm. Bull. (Tokyo), 52: 818-829 (2004)); 1-benzothiepine 1,1-dioxide and 1-benzazepine derivatives containing a tertiary amine moiety (Seto et al., Chem. Pharm. Bull. (Tokyo), 52: 577-590 (2004)); N-[3-(4-benzylpiperidin-1-yl)propyl]-N,N′-diphenylureas (Imamura et al., Bioorg. Med. Chem., 12: 2295-2306 (2004)); 5-oxopyrrolidine-3-carboxamide derivatives (Imamura et al., Chem. Pharm. Bull. (Tokyo), 52: 63-73 (2004); anilide derivatives with a quaternary ammonium moiety (Shiraishi et al., J. Med. Chem., 43: 2049-2063 (2000)); AK602/0N04128/GW873140 (Nakata et al., J. Virol., 79: 2087-2096 (2005)); spirodiketopiperazine derivatives (Maeda et al., J. Biol. Chem., 276: 35194-35200 (2001); Maeda et al., J. Virol., 78: 8654-8662 (2004)); and selective CCR5 antagonists (Thoma et al., J. Med. Chem., 47: 1939-1955 (2004)).

In one embodiment, the CCR5 binding agent, such as PRO 140, is administered in combination with one or more of SCH-C, SCH-D (SCH 417670, or vicriviroc), UK-427,857 (maraviroc), GW873140, TAK-652, TAK-779 AMD070, or AD101. See U.S. Pat. No. 8,821,877.

In one embodiment, the competitive binding agent to a CCR5 cell receptor, such as PRO 140, exhibits synergistic effects when administered in combination with one or more other therapeutic molecules or treatment, such as a cellular therapy, a small molecule, a chemotherapeutic, or an inhibitor of CCR5/CCL5 signaling. “Synergy” between two or more agents refers to the combined effect of the agents which is greater than their additive effects. Synergistic, additive, or antagonistic effects between agents may be quantified by analysis of the dose-response curves using the Combination Index (CI) method. A CI value greater than 1 indicates antagonism; a CI value equal to 1 indicates an additive effect; and a CI value less than 1 indicates a synergistic effect. In one embodiment, the CI value of a synergistic interaction is less than 0.9. In another embodiment, the CI value is less than 0.8. In another embodiment, the CI value is less than 0.7.

In several embodiments, preventing the cancer comprises reducing the number of circulating tumor cells, epithelial mesenchymal transition cells, and/or cancer associated macrophage-like cells. As used herein, “circulating tumor cell” (CTC) refers to cancer cells that have detached from the tumor and begun to circulate in the vasculature and lymphatics; CTCs serve as precursors to metastatic cancer. As used herein, “epithelial-mesenchymal transition cell” (EMT cells), refers to epithelial cells that have undergone transdifferentiation into motile mesenchymal cells. Events undergone by epithelial cells during the EMT transdifferentiation process may include, but are not limited to, the dissolution of the epithelial cell-cell junctions; alterations to polarity; reorganization of the cytoskeletal architecture and changes in cell shape; downregulation of an epithelial gene expression signature and activation mesenchymal phenotype-defining genes; increased cell protrusions and motility; enhanced invasive capability; acquired resistance to senescence and apoptosis. Finally, as used herein, “cancer associated macrophage-like cell” (CAML) refers to a highly differentiated giant circulating (macrophage-like) cell that exhibits CD14+ expression and vacuoles of phagocytosed material; CAMLs are isolated from the peripheral blood of patients with cancer, including, but not limited to, breast, prostate, or pancreatic cancer.

SEQUENCE LISTING

>SEQ ID NO: 1 VL protein sequence; signal peptide at amino acids 1-19; CDRs underlined
MKLPVRLLVLMFWIPASSSDIVMTQSPLSLPVTPGEPASISCRSSQRLLSSYGHTYLHWY
LQKPGQSPQLLIYEVSNRFSGVPDRFSGSGSGTDFTLKISRVEAEDVGVYYCSQSTHVPL
TFGQGTKVEIK
>SEQ ID NO: 2 VL nucleotide sequence
>SEQ ID NO: 3 PRO#2 VH protein sequence; signal peptide at amino acids 1-19; CDRs
underlined
MEWSGVFIFLLSVTAGVHSEVQLVESGGGLVKPGGSLRLSCAASGYTFSNYWIGWVRQ
APGKGLEWIGDIYPGGNYIRNNEKFKDKTTLSADTSKNTAYLQMNSLKTEDTAVYYCG
SSFGSNYVFAWFTYWGQGTLVTVSS
>SEQ ID NO: 4 PRO#2 VH nucleotide sequence
>SEQ ID NO: 5 PRO#1 VH protein sequence; signal peptide at amino acids 1-19; CDRs
underlined
MEWSGVFIFLLSVTAGVHSQVQLVQSGPDVKKPGTSMKMSCKTSGYTFSNYWIGWVR
QAPGQGLEWIGDIYPGGNYIRNNEKFKDKTTLTADTSTSTAYMQLGSLRSEDTAVYYCG
SSFGSNYVFAWFTYWGQGTLVTVSS
>SEQ ID NO: 6 PRO#1 VH nucleotide sequence
>SEQ ID NO: 7 heavy chain protein sequence
EVQLVESGGG LVKPGGSLRL SCAASGYTFS NYWIGWVRQA PGKGLEWIGD
IYPGGNYIRNNEKFKDKTTL SADTSKNTAY LQMNSLKTED TAVYYCGSSF
GSNYVFAWFT YWGQGTLVTVSSASTKGPSV FPLAPCSRST SESTAALGCL
VKDYFPEPVT VSWNSGALTS GVHTFPAVLQSSGLYSLSSV VTVPSSSLGT
KTYTCNVDHK PSNTKVDKRV ESKYGPPCPS CPAPEFLGGPSVFLFPPKPK
DTLMISRTPE VTCVVVDVSQ EDPEVQFNWY VDGVEVHNAK TKPREEQFNS
TYRVVSVLTV LHQDWLNGKE YKCKVSNKGL PSSIEKTISK AKGQPREPQV
YTLPPSQEEMTKNQVSLTCL VKGFYPSDIA VEWESNGQPE NNYKTTPPVL
DSDGSFFLYS RLTVDKSRWQEGNVFSCSVM HEALHNHYTQ KSLSLSLGK
>SEQ ID NO: 8 light chain protein sequence
DIVMTQSPLS LPVTPGEPAS ISCRSSQRLL SSYGHTYLHW YLQKPGQSPQ
LLIYEVSNRFSGVPDRFSGS GSGTDFTLKI SRVEAEDVGV YYCSQSTHVP
LTFGQGTKVE IKRTVAAPSVFIFPPSDEQL KSGTASVVCL LNNFYPREAK
VQWKVDNALQ SGNSQESVTE QDSKDSTYSLSSTLTLSKAD YEKHKVYACE
VTHQGLSSPV TKSFNRGEC
>SEQ ID NO: 9 LCDR1 amino acid sequence
RSSQRLLSSYGHTYLH
>SEQ ID NO: 10 LCDR2 amino acid sequence
EVSNRFS
>SEQ ID NO: 11 LCDR3 amino acid sequence
SQSTHVPLT
>SEQ ID NO: 12 HCDR1 amino acid sequence
NYWIG
>SEQ ID NO: 13 HCDR2 amino acid sequence
DIYPGGNYIRNNEKEKD
>SEQ ID NO: 14 HCDR3 amino acid sequence
SFGSNYVFAWFTY
>SEQ ID NO: 15 Homo sapiens CCR5, NCBI Reference Sequence: NP_000570.1
MDYQVSSPIYDINYYTSEPCQKINVKQIAARLLPPLYSLVFIFGFVGNMLVILILINCKRLK
SMTDIYLLNLAISDLFFLLTVPFWAHYAAAQWDFGNTMCQLLTGLYFIGFFSGIFFIILLTI
DRYLAVVHAVFALKARTVTFGVVTSVITWVVAVFASLPGIIFTRSQKEGLHYTCSSHFPY
SQYQFWKNFQTLKIVILGLVLPLLVMVICYSGILKTLLRCRNEKKRHRAVRLIFTIMIVYF
LFWAPYNIVLLLNTFQEFFGLNNCSSSNRLDQAMQVTETLGMTHCCINPIIYAFVGEKFR
NYLLVFFQKHIAKRFCKCCSIFQQEAPERASSVYTRSTGEQEISVGL

### EXAMPLES

### Example 1

### Leronlimab Inhibits Tumor Growth in Mice

Only a small subpopulation of cells within a breast tumor are capable of initiating tumor formation in mice. These tumor-initiating cells correlate with an increased propensity to metastasize. CCR5+ breast cancer epithelial cells were shown to form mammospheres and initiate tumors with >60-fold greater efficiency in mice [Jiao 2018]. Experiments with stably transfected SUM-159 breast cancer cells with an expression vector encoding CCR5 showed that both endogenous CCR5 and overexpression of CCR5 in breast cancer cells is sufficient for the induction of basal breast cancer cellular tumor formation in vivo [Jiao 2018].

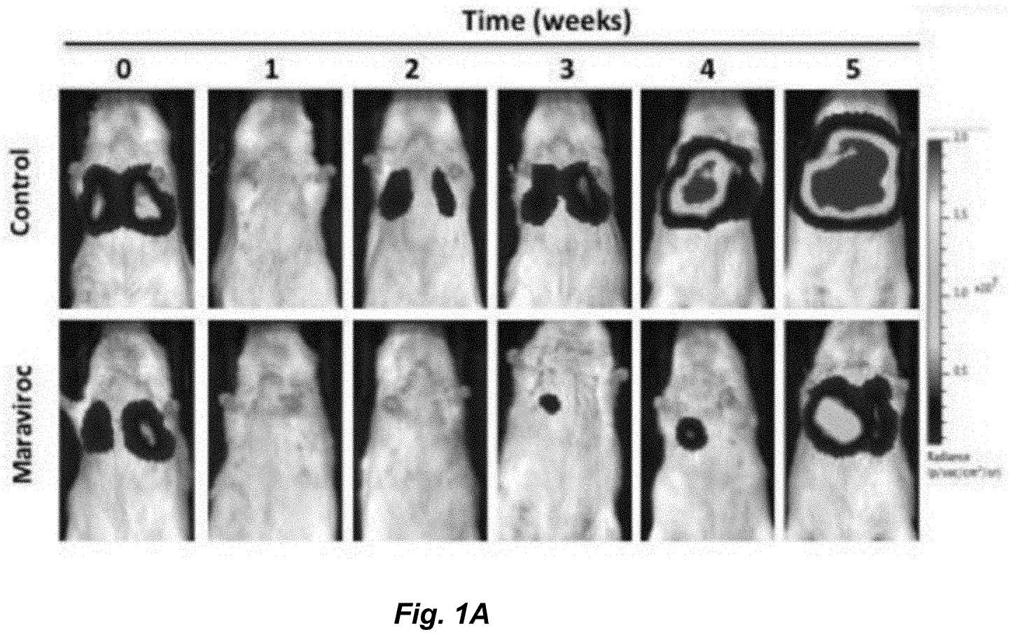

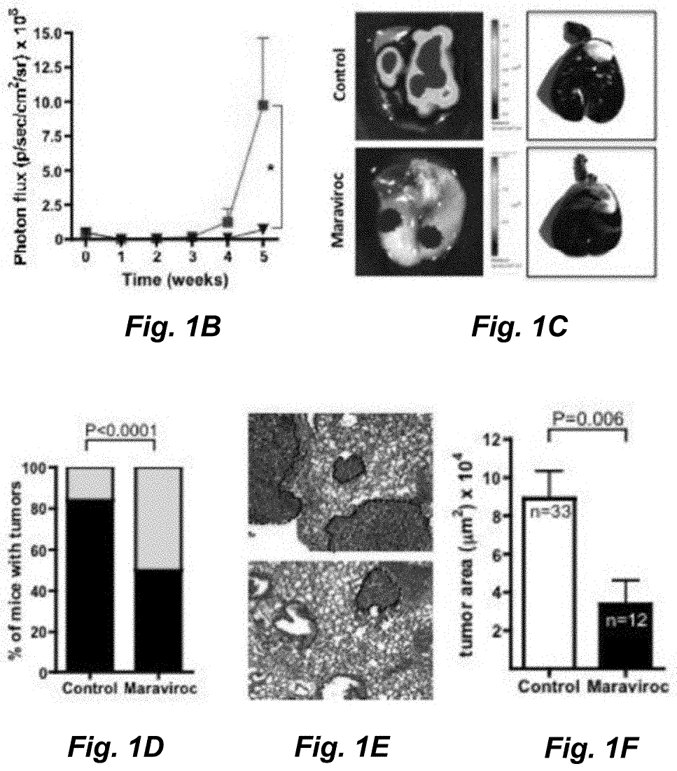

CCR5 has been shown to be sufficient to induce in vitro invasiveness and metastasis of breast cancer cells that is blocked by CCR5 inhibitors [Velasco-Velazquez]. The CCR5 inhibitor maraviroc was shown to block homing of breast cancer cells to the lungs (FIG. 1). The dose of CCR5 inhibitor used in these mouse models was the same as the dose used in patients for HIV treatment. Preclinical studies have also demonstrated that oncogenic transformation of immortal human breast cancer cells, with either Ha-Ras, c-Myc, ErbB2 (NeuT) or c-Src, induces the mRNA expression and protein abundance of CCR5 during the process of transformation [Velasco-Velazquez].

To determine the growth inhibitory effect of leronlimab (PRO 140) and compare its effect with FDA-approved CCR5 antagonists maraviroc and vicriviroc, preclinical studies were carried out in female NCI Athymic NCr-nu/nu mice. Each mouse received one million (10⁶) MDA-MB-231 cells expressing Luc2-eGFP (called MDA-MB-231.pFLUG) through the tail vein. Mice were treated by oral gavage feeding with maraviroc (8 mg/kg twice a day), vicriviroc (16 mg/kg twice a day), or by intraperitoneal injection of leronlimab (PRO 140) (2 mg/mouse, twice a week). Treatment was started one day before the injection.

In vivo bio-luminescence imaging was performed following intraperitoneal injection with 100 μl of D-luciferin (30 mg/mL) to the control and treated groups. FIG. 1A, FIG. 1B, FIG. 1C, FIG. 1D, FIG. 1E, and FIG. 1F show maraviroc inhibition of lung metastasis in a mouse model. FIG. 1A shows timecourse images of mouse lung metastasis for a mouse treated with maraviroc. FIG. 1B shows photon flux measurements taking weekly during the timecourse. FIG. 1C shows the presence of pulmonary tumors. FIG. 1D is a plot of the percentage of mice with tumors. FIG. 1E shows histologic staining of the area of the slide covered in tumors. FIG. 1F shows tumor area.

### Example 2

### CCR5 Expression in Patient Samples

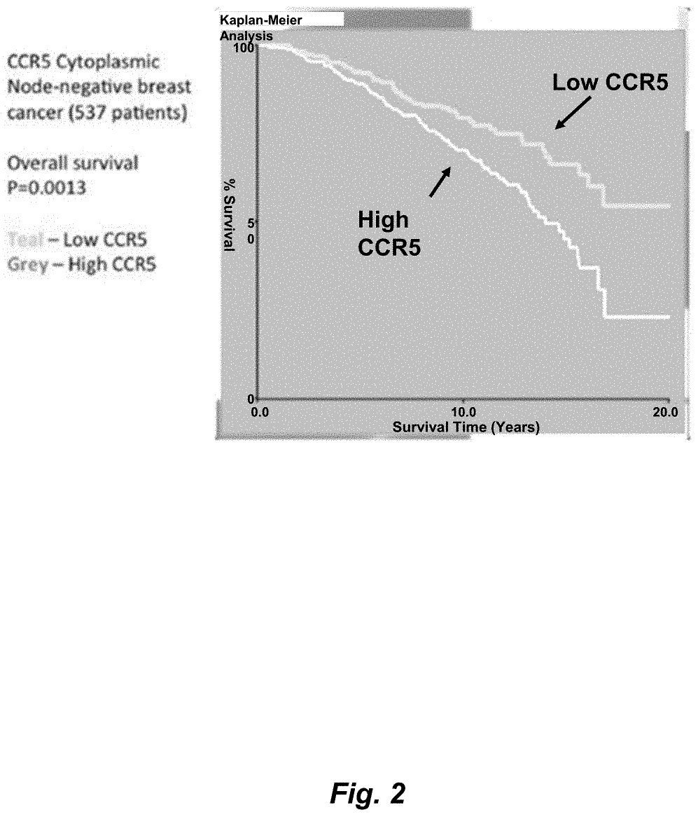

The correlation of CCR5 expression in human breast cancer versus patient outcome was evaluated, as shown in FIG. 2. Immunohistochemical staining for CCR5 was conducted in samples from 537 patients with node-negative breast cancer, and survival was plotted for patients whose samples showed low CCR5 expression, and for patients whose samples shows high CCR5 expression. As shown in FIG. 2, high CCR5 expression correlates with poor survival.

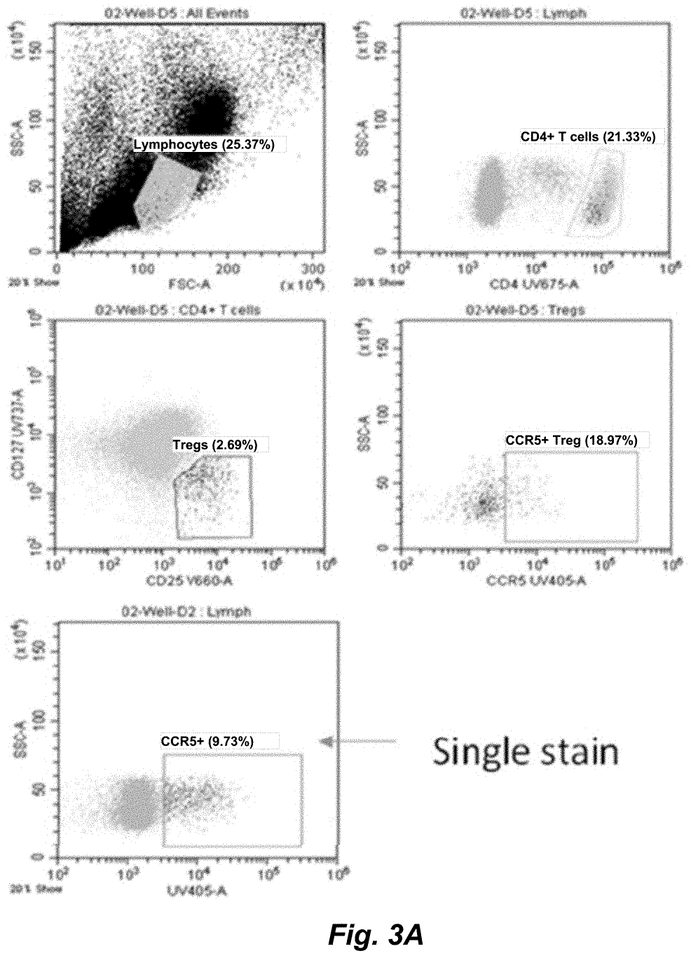

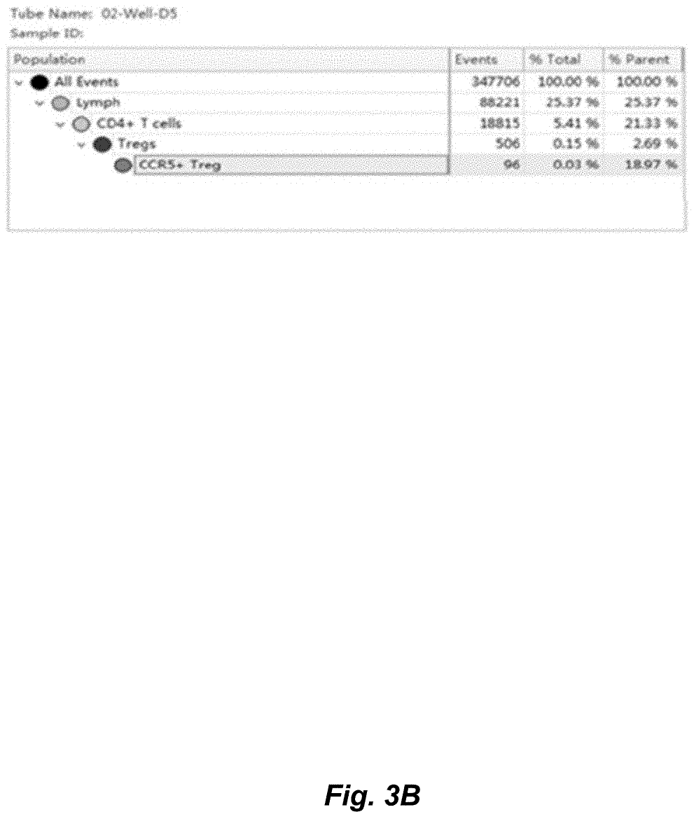

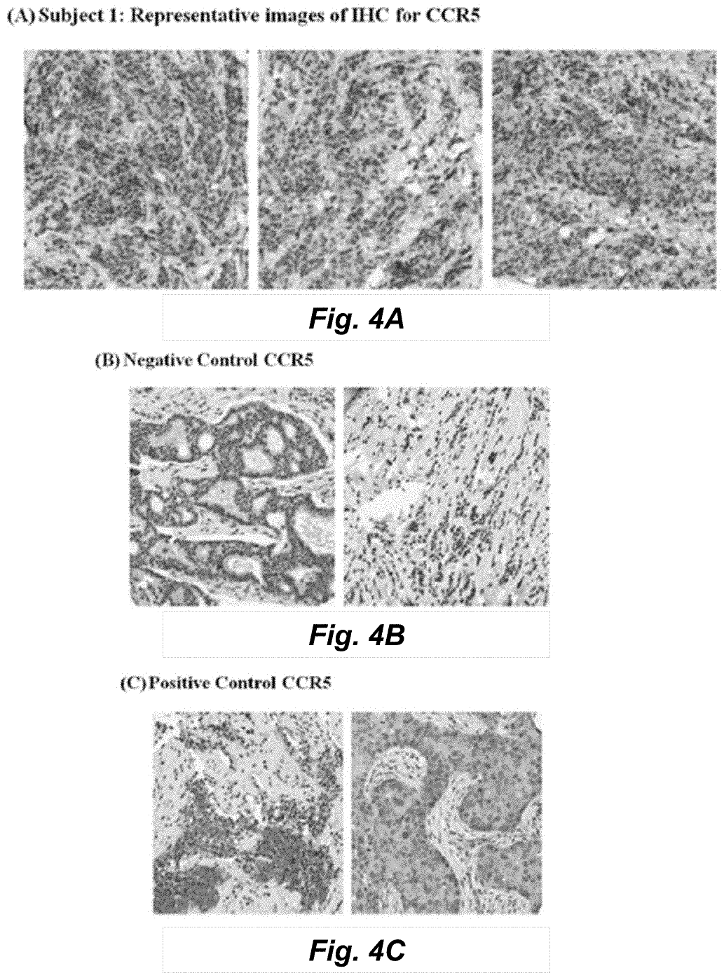

The role of CCR5 blockade of the CCL5-CCR5 pathway in immune control of tumors has been defined in several publications in the peer-reviewed medical literature [Manes, 2003]. CCR5 expression on tumor cells, especially those that evade local immune control in the primary tumor, leads to CCR5-positive circulating tumor cells that have the capability to disseminate and migrate into distant tumor sites again through the CCL5-CCR5 axis. Previous research and current data has also identified other immune mediated anti-tumor effects from CCR5 blockade [Lanitis, 2017, Halama, 2016]. Previous published reports suggest CCR5 is expressed by Treg cells which migrate into tumors due to the expression of CCL5 by lymphocytes [de Oliveira, 2017, Del Prete, 2017, Lanitis, 2017]. Tregs are responsible for minimizing or eliminating the anti-tumor effects of CD8 T cells that are restored by blockade of PD-L1/PD-1 by the new class of immune-oncology drugs [de Oliveira, 2017]. Further, blocking CCR5 on tumor-associated macrophages (TAMS), one of the major cells in the tumor microenvironment that suppresses the T-cell mediated anti-tumor immune response, restores anti-tumor activity by re-programming the TAMs [Lanitis, 2017, Walens, 2019]. Data from a novel 24-color flow cytometry assay performed on single cell suspensions created with the IVD IncellPREP device, confirmed the expression of CCR5 on Tregs from the tumor microenvironment in lung, breast, and bladder cancer samples (FIG. 3A and FIG. 3B). This technology or CCR5 immunohistochemistry (IHC) of biopsies already obtained has allowed for the selection of patients that harbor CCR5-expressing tumor cells as well CCR5-expressing intra-tumor immune cells in the tumor microenvironment. FIG. 4A, FIG. 4B, and FIG. 4C show immunohistochemical staining for CCR5 in triple negative breast cancer biopsies form a first subject with triple negative breast cancer.

### Example 3

### Leronlimab and Carboplatin Treatment of CCR5+ Metastatic TNBC

A phase Ib/II study of leronlimab (PRO 140) combined with carboplatin in patients with CCR5+ metastatic Triple Negative Breast Cancer (mTNBC) is ongoing. The primary objective of Phase 1b is to determine the safety, tolerability, and maximum tolerated dose (MTD) of PRO 140 in patients with TNBC, when combined with carboplatin to define a recommended Phase II dose of the combination. The primary objective of phase 2b is to evaluate the impact on progression-free survival (PFS) of the combination of PRO 140 and carboplatin in patients with CCR5+ TNBC previously treated with anthracyclines and taxanes in a neoadjuvant and adjuvant setting. For further investigational plan details please refer to section 5.3.5 in SNO01 (IND 141723).

A first subject enrolled in the study, Patient D, is a 42 year old female with Stage IV metastatic triple negative breast cancer. Subject has a history of left breast cancer with a right lung metastasis.

The subject was diagnosed with Stage IIA Grade 3 Invasive Ductal Carcinoma (ER neg/PR neg/HER-2-NEU neg. and previously received dose-dense Adriamycin (Doxorubicin) and Cyclophosphamide [ddAC] and Paclitaxel. The subject underwent a left lumpectomy of the breast and a sentinel lymph node biopsy three weeks following diagnosis.

The subject signed the pre-screening informed consent for the Protocol CD07_TNBC ten weeks following diagnosis.

The baseline target lesion was identified in the right upper lung at the size of 25 mm. The lesion was described as a pleural-based, major fissure, soft tissue density nodule in the right hilum.

Approximately six weeks following the identification and measurement of the baseline lesion, the subject received the first treatment of 350 mg leronlimab (PRO 140) (1). Each treatment cycle consisted of 21 days. Leronlimab (PRO 140) was administered subcutaneously weekly on Days 1, 8, and 15 in combination with carboplatin AUC 5 on Day 1 of each cycle (C) (every 21 days). This treatment regimen was used for all subjects enrolled in the mTNBC study, unless otherwise indicated.

TABLE 1
Leronlimab (PRO 140) and Carboplatin Doses
Patient D

| Visit | Study Treatment Administration |
|---|---|
| Pre-Screening | NA |
| Screening | NA |
| NA | Carboplatin 500 mg |
| C1D1 | Leronlimab (PRO 140) 350 mg |
| C1D8 | Leronlimab (PRO 140) 350 mg |
| C1D15 | Leronlimab (PRO 140) 350 mg |
| C2D1 | Leronlimab (PRO 140) 350 mg |
| | Carboplatin 500 mg |
| C2D8 | Leronlimab (PRO 140) 350 mg |
| C2D15 | Leronlimab (PRO 140) 350 mg |
| C3D1 | Leronlimab (PRO 140) 350 mg |
| | Carboplatin 500 mg |
| C3D8 | Leronlimab (PRO 140) 350 mg |
| C3D15 | Leronlimab (PRO 140) 350 mg |
| C4D1 | Leronlimab (PRO 140) 350 mg |
| | Carboplatin 250 mg |
| C4D8 | Leronlimab (PRO 140) 350 mg |
| C4D15 | Leronlimab (PRO 140) 350 mg |
| C5D1 | Leronlimab (PRO 140) 350 mg |
| | Carboplatin 600 mg |
| C5D8 | Leronlimab (PRO 140) 350 mg |
| C5D15 | Leronlimab (PRO 140) 350 mg |
| C6D1 | Leronlimab (PRO 140) 350 mg |
| | Carboplatin* |

*Pending dose information

The blood sample for circulating tumor cells (CTC) and cancer-associated macrophage-like cells (CAMLs) assessment was collected at baseline and subsequently at Day 1 of each treatment cycle to assess changes in CTCs and CAMLs after treatment and to perform correlative analysis between CCR5 expression and PD-L1 expression.

Creatv Microtech has developed a size-based technology and detection methodology (LifeTrac Assay) that enables the collection and characterization of all cancer-associated cells in the blood i.e., CTCs, epithelial mesenchymal transition cells (EMTs) and CAMLs [Adams Cytometry 2015, Adams RSC 2014]. The CellSieve™ filtration platform is used to capture CAMLs and CTCs.

The summary of results for CCR5 expression and PD-L1 expression is as follows:

TABLE 2
Patient D- CCR5-expressing and PD-L1-expressing
CTCs, EMTs, and CAMLs Result

| | Date of Blood Draw | | | | | |
|---|---|---|---|---|---|---|
| | Baseline | C1D1 | C2D1 | C3D1 | C4D1 | C5D1 |
| CCR5 | | | | | | |
| Number of CTCs | 1 | 0 | 0 | 0 | 0 | 0 |
| Number of | 1 | 0 | 0 | 0 | 0 | 0 |
| Apoptotic CTCs | | | | | | |
| Number of EMTs | 1 | 1 | 0 | 0 | 0 | 0 |
| Number of CAMLs | 1 | 0 | 1 | 3 | 0 | 1 |
| Largest CAML | 0 | A | 7 | 9 | μm | 3 |
| | μm | | μm | μm | | μm |
| PD-L1 | | | | | | |
| Number of CTCs | 0 | 0 | 0 | 0 | 0 | 0 |
| Number of | 3 | 0 | 0 | 0 | 0 | 0 |
| Apoptotic CTC | | | | | | |
| Number of EMTs | 1 | 1 | 0 | 0 | 0 | 0 |
| Number of CAMLs | 1 | 1 | 2 | 1 | 1 | 2 |
| Largest CAML | 50 | 47 | 69 | 30 | 31 | 56 |
| | μm | μm | μm | μm | μm | μm |

The summary for results of total CTCs, EMTs, and CAMLs is as follows:

TABLE 3
Patient D -CTCs, EMTs, and CAMLs Results

| | Baseline | C1D1 | C2D1 | C4D1 | C5D1 | C6D1 |
|---|---|---|---|---|---|---|
| CTC-Total | 5 | 0 | 0 | 0 | 0 | 0 |
| EMT-Total | 2 | 2 | 0 | 0 | 0 | 0 |
| CAML- Total | 2 | 1 | 3 | 1 | 3 | 8 |

Scans were taken at the end of every two cycles (every 6 weeks). The subject had Scan 1 after six weeks, Scan 2 after 12 weeks, and Scan 3 after 18 weeks (Table 4). At scan 3, there were no new lung nodules found. The target lesion found on the right upper lobe of the lung nodule measured 2.1×1.6 cm, which was previously 2.4×1.9 (on 28 Oct. 2019), had a 20% decrease in size.

TABLE 4
Patient D - Tumor imaging
Patient D

| | Target Lesion | | |
|---|---|---|---|
| | (Right Upper Lobe | | |
| | lung nodule) | | Comments |
| Baseline Scan | 25 | mm | |
| Scan 2 | 2.4 × 1.9 | cm | |
| Scan 3 | 2.1 × 1.6 | cm | Right lung metastasis demonstrates |
| | | | maximum standardized uptake |
| | | | values (SUVs) of 6.8 (previously |
| | | | 15.3). Previously identified right |
| | | | hilar lymph node resolved. No new |
| | | | lymphadenopathy or metastatic |
| | | | disease reported on the diagnostic |
| | | | CT chest, abdomen and pelvis. |

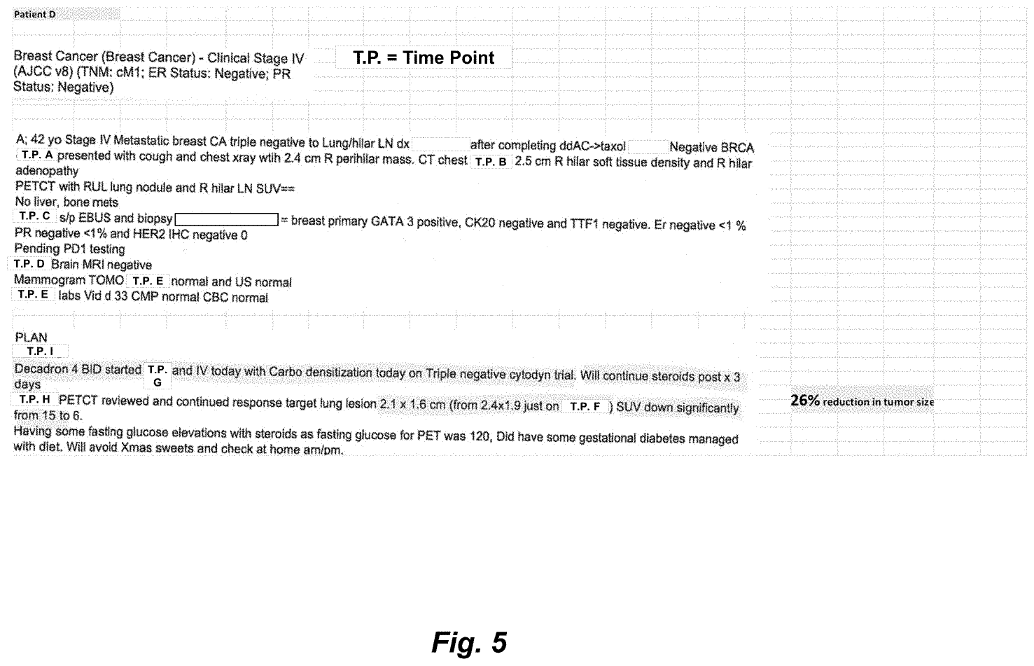

At the time that the subject had completed the Cycle 6 Day 1 visit, the subject had been receiving weekly injections of leronlimab (PRO 140) and a carboplatin infusion every three weeks per protocol. At the time of the Cycle 6 Day 1 visit, no serious adverse events had been reported. The adverse events reported are shown in FIG. 5.

Following 16 weeks of leronlimab treatment of the first subject enrolled under the mTNBC study showed no detectable circulating tumor cells (CTC) or putative metastatic tumor cells in the peripheral blood. Furthermore, the patient had large reductions in CCR5 expression on cancer-associated cells after approximately 11 weeks of treatment with leronlimab. Additionally, the target lesion found on the right upper lobe of the lung nodule showed a greater than 20% decrease in size (as measured by tumor volume). This result was a remarkable improvement in disease outcome and demonstrates that leronlimab is a promising adjuvant therapy for the treatment of metastatic triple negative breast cancer.

A second subject, Patient C, with mTNBC was enrolled in the mTNBC study. Data collected from the second patient enrolled in the Company's mTNBC Phase 1b/2 trial showed no detectable levels of CTC after two weeks of treatment with the previously described treatment regimen of leronlimab in combination with carboplatin. This patient also showed a 70% reduction in EMT cells after just two weeks of treatment. Initial data from the second patient in the mTNBC trial indicated the CTC dropped to zero after two weeks of treatment with leronlimab. Additionally, the second patient had an initial CAML count of 45, and following at least two weeks of treatment the CAML count decreased to 30.

A third subject was enrolled in the mTNBC study. CTC+EMT counts were measured at initiation of treatment and two weeks following initiation of treatment with the previously described treatment regimen. The results indicate that the third patient's total CTC+EMT counts decreased by 75% during the first two weeks of treatment.

### Example 4

### Leronlimab Treatment of Metastatic HER2+ Breast Cancer

This subject, Patient A, is a 78-year-old female with a diagnosis of metastatic breast cancer, stage IV. The subject previously received Taxotere/Herceptin/Pertuzumab as frontline therapy for metastatic HER2 positive breast cancer. She had partial response for her systemic disease, but then developed diffuse brain metastases (systemic disease stable). She completed whole-brain radiation therapy and continues on Herceptin and Pertuzumab. She has neuropathy and residual side effects from chemotherapy, which limits use of current second-line options due to concern for side effects. Leronlimab (PRO 140) was requested in an attempt to achieve disease control and prolong chemotherapy-free interval as this patient may not be able to tolerate chemotherapy side effects.

The subject is receiving weekly injections of 700 mg leronlimab (PRO 140) (Table 5).

TABLE 5
Leronlimab (PRO 140) Administration Schedule

Single Patient Emergency Use IND Subject

| Visit | Date | Study Treatment Administration |
|---|---|---|
| Screening | | NA |
| Treatment 1 | DAY 1 | Leronlimab (PRO 140) 700 mg |
| Treatment 2 | DAY 10 | Leronlimab (PRO 140) 700 mg |
| Treatment 3 | DAY 17 | Leronlimab (PRO 140) 700 mg |
| Treatment 4 | DAY 24 | Leronlimab (PRO 140) 700 mg |
| Treatment 5 | DAY 35 | Leronlimab (PRO 140) 700 mg |
| Treatment 6 | DAY 46 | Leronlimab (PRO 140) 700 mg |

Approximately four weeks following the initial treatment, a CT scan was conducted and the results indicated no signs of new metastatic spots in the liver, lung and brain during the treatment with leronlimab, as compared to the CT scan results obtained approximately 6 weeks prior to the initiation of treatment.

Approximately two months following the initial treatment, no new metastasis was detectable in the brain after treatment with leronlimab being the only treatment the subject was receiving to treat brain metastasis. Prior to enrolling in the trial, the patient had 18 identifiable tumor spots in the brain. At approximately two months following the start of weekly 700 mg doses of leronlimab, only three lesions were identifiable, as detected by Mill. Furthermore, the treatment resulted in a 56% reduction in tumor volume of the largest brain tumor identified in the subject's brain at the initiation of treatment.

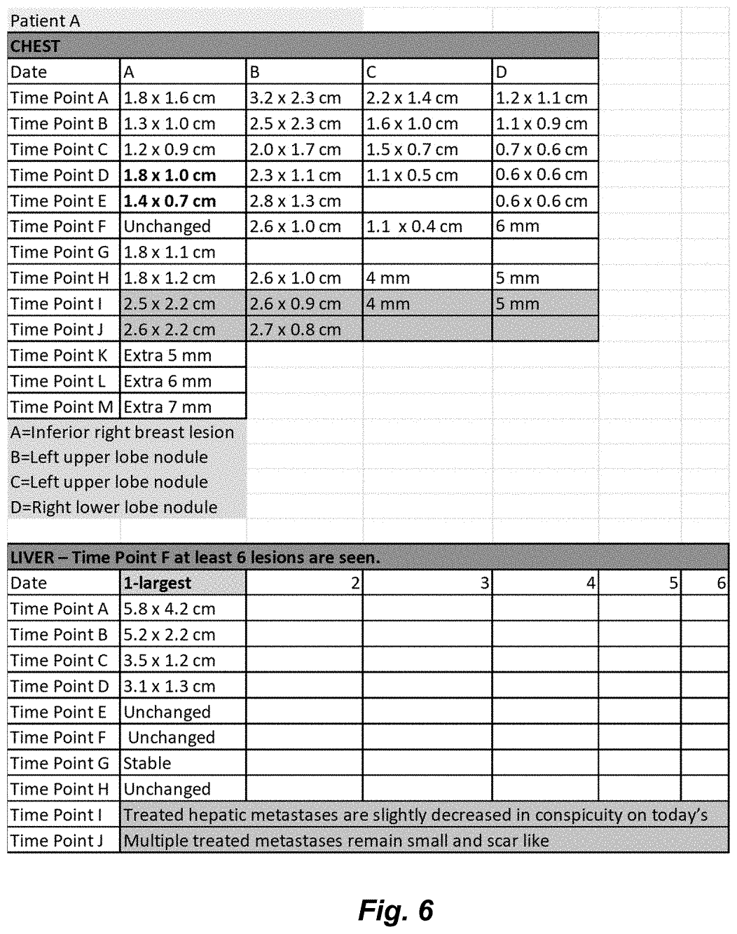

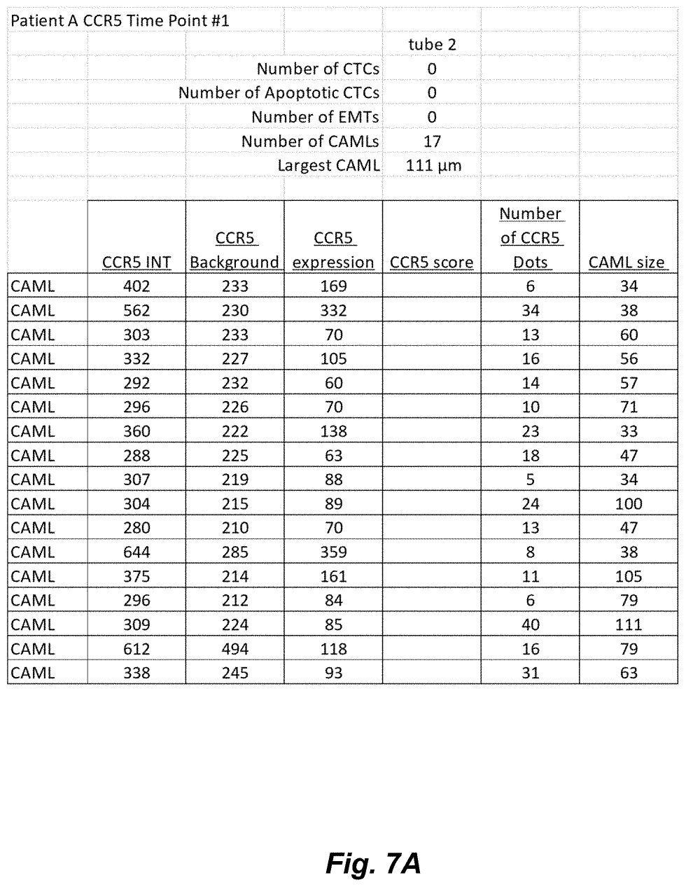

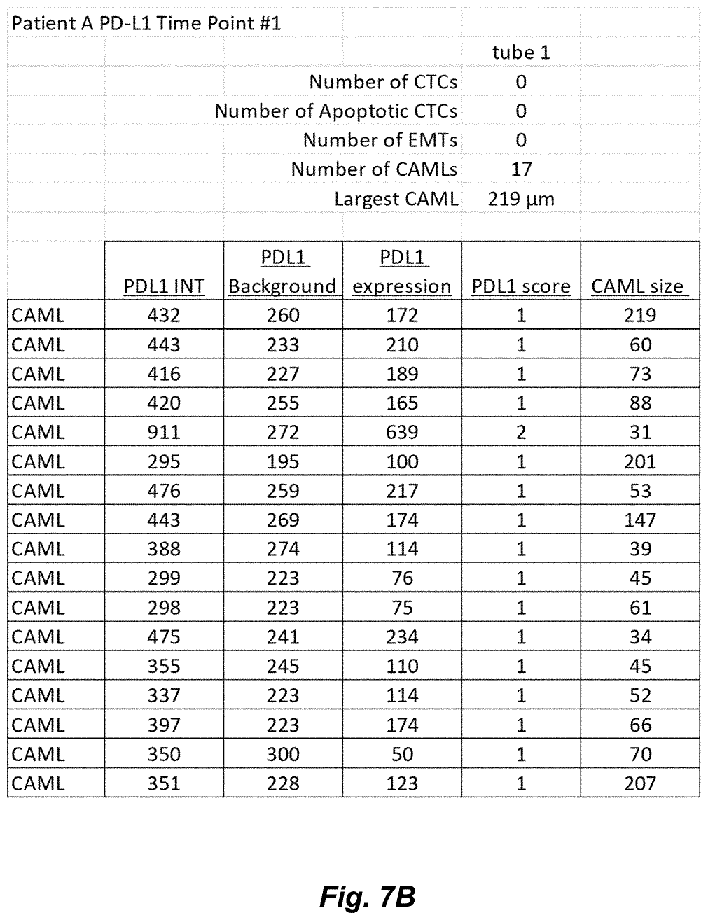

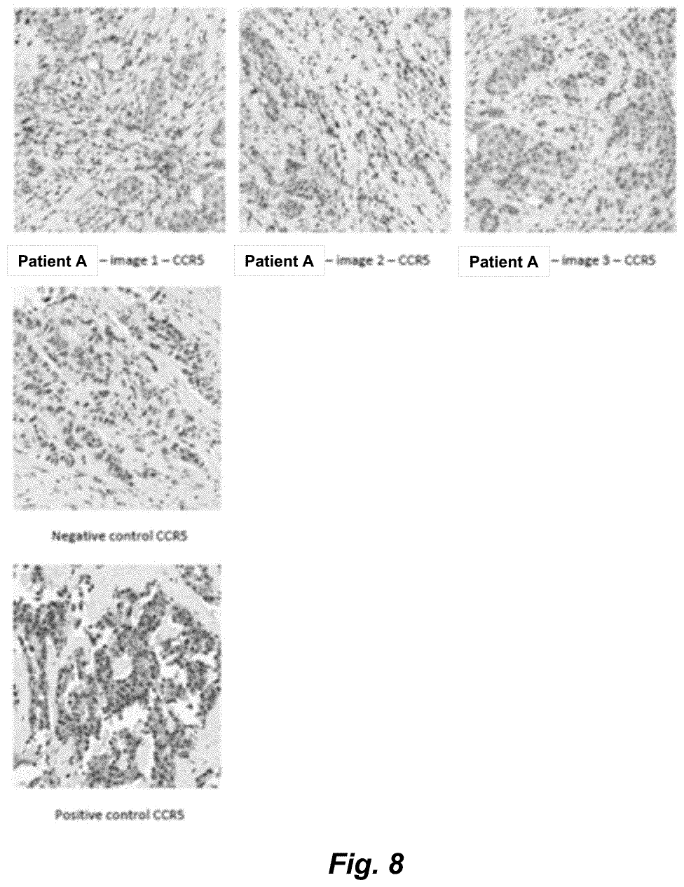

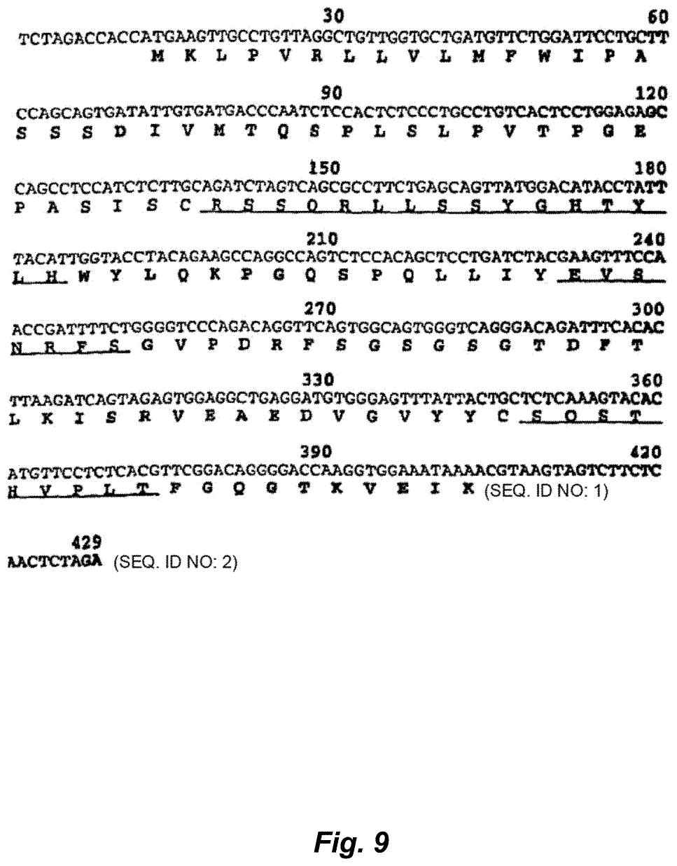

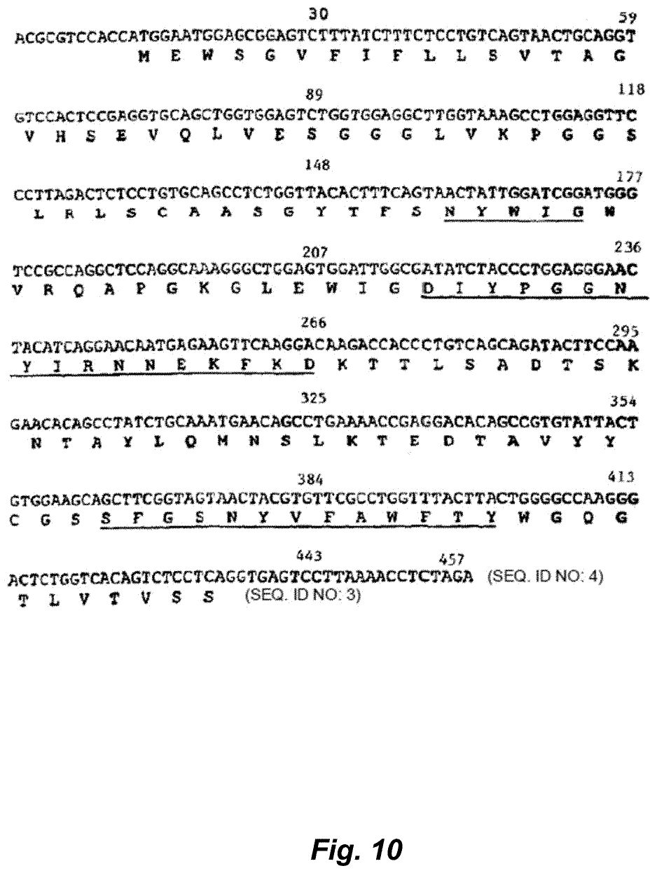

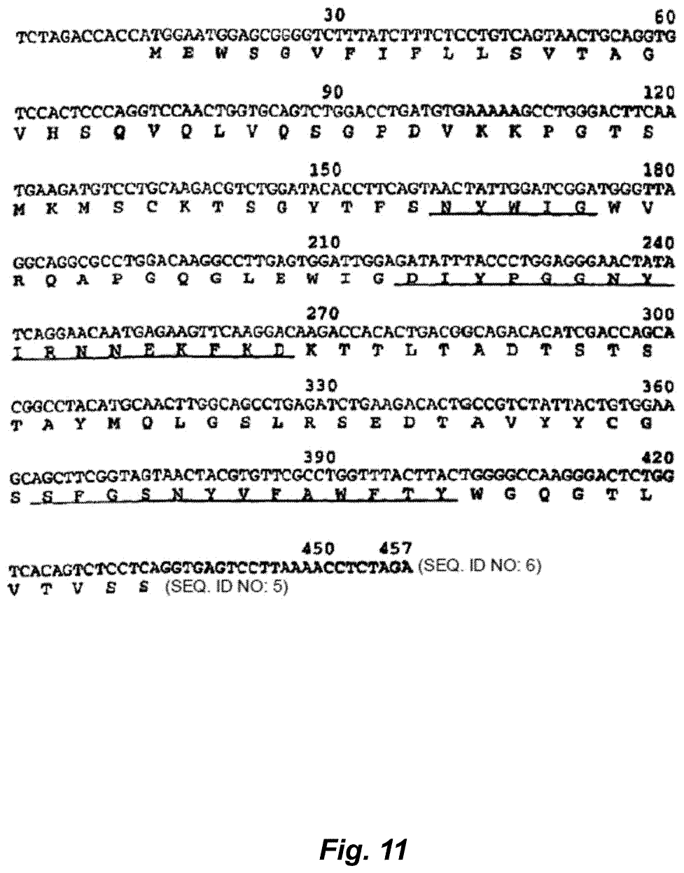

Approximately ten weeks following the initiation of treatment, the subject's CTC and EMT counts were measured, and zero CTCs and zero EMTs were identified. Lesion and nodule sizes were measured in the breast and liver of Patient A and metastases were also qualitatively described (FIG. 6). Protein expression levels of CCR5 (FIG. 7A) and PD-L1 (FIG. 7B) on individual CAMLs from Patient A were measured by flow cytometry and reported as Mean Fluorescence Intensity (MFI). CCR5 MFI (“CCR5 INT”) was calculated by subtracting background signal of a negative control sample from the experimental value. CAML size was also measured and reported in μM. The subject's tumor biopsy showed high CCR5 expression on tumor infiltrating leukocytes (FIG. 8).

### Example 5

### Leronlimab for Treatment of Solid Tumors

A Phase 2 protocol for a basket trial with the U.S. Food and Drug Administration (FDA) as an Investigational New Drug (IND) Application for the treatment of cancer is ongoing. At least 22 solid tumor cancer types are being treated under this protocol, including, but not limited to, melanoma, brain (glioblastoma), throat, lung, stomach, colon, colon carcinoma, breast, testicular, ovarian, uterine, pancreas, bladder, esophageal, appendix, and prostate cancers, among other indications. The basket trial is a Phase 2 study with 30 patients with CCR5+ locally advanced or metastatic solid tumors. Leronlimab will be administered subcutaneously as a weekly dose of 350 mg. Subjects participating in this study will be allowed to receive and continue the standard-of-care chemotherapy as determined by the treating physician.

Several patients have been enrolled in the Phase 2 basket trial to date. Patients were diagnosed with breast, colon, esophageal, appendix, ovarian, or prostate cancers prior to enrollment in the study.

All of the U.S. patents, U.S. patent application publications, U.S. patent applications, foreign patents, foreign patent applications referred to in this specification and/or listed in the Application Data Sheet, including U.S. Provisional Patent Application No. 62/960,613 filed on Jan. 13, 2020, U.S. Provisional Patent Application No. 62/968,954 filed on Jan. 31, 2020 and U.S. Provisional Patent Application No. 62/977,023 filed on Feb. 14, 2020, are incorporated herein by reference, in their entirety. Aspects of the embodiments can be modified, if necessary to employ concepts of the various patents and applications to provide yet further embodiments. The various embodiments described above can be combined to provide further embodiments.

While specific embodiments of the invention have been illustrated and described, it will be readily appreciated that the various embodiments described above can be combined to provide further embodiments, and that various changes can be made therein without departing from the spirit and scope of the invention. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

Adams D L, Zhu P, Makarova O V, Martin S S, Charpentier M, Chumsri S, Li S, Amstutz P, Tang C M. The systematic study of circulating tumor cell isolation using lithographic microfilters. RSC advances. 2014; 4(9):4334-42.
Adams D L, Stefansson S, Haudenschild C, Martin S S, Charpentier M, Chumsri S, Cristofanilli M, Tang C M, Alpaugh R K. Cytometric characterization of circulating tumor cells captured by microfiltration and their correlation to the Cellsearch® CTC test. Cytometry Part A. 2015 February; 87(2):137-44.
Aysola K, Desai A, Welch C, Xu J, Qin Y, Reddy V, Matthews R, Owens C, Okoli J, Beech D J, Piyathilake C J, Reddy S P, Rao V N. Triple Negative Breast Cancer—An Overview. Hereditary Genet. 2013; 2013 (Suppl 2). pii: 001. PubMed PMID: 25285241; PubMed Central PMCID: PMC4181680.
Dawood S, Hu R, Homes M D, Collins L C, Schnitt S J, Connolly J, et al. Defining breast cancer prognosis based on molecular phenotypes: results from a large cohort study. Breast Cancer Res Treat. 2011 February; 126(1):185-92.
de Oliveira C E, Gasparoto T H, Pinheiro C R, Amor N G, Nogueira M R, Kaneno R, Garlet G P, Lara V S, Silva J S, Cavassani K A, Campanelli A P. CCR5-dependent homing of T regulatory cells to the tumor microenvironment contributes to skin squamous cell carcinoma development. Molecular cancer therapeutics. 2017 Dec. 1; 16(12):2871-80.
Del Prete A, Schioppa T, Tiberio L, Stabile H, Sozzani S. Leukocyte trafficking in tumor microenvironment. Current opinion in pharmacology. 2017 Aug. 1; 35:40-7.
Dent R, Trudeau M, Pritchard K I, Hanna W M, Kahn H K, Sawka C A, Lickley L A, Rawlinson E, Sun P, Narod S A. Triple-negative breast cancer: clinical features and patterns of recurrence. Clin Cancer Res. 2007 Aug. 1; 13 (15 Pt 1): 4429-34.PubMed PMID: 17671126.
Foulkes W D, Smith I E, Reis-Filho J S. Triple-negative breast cancer. New England journal of medicine. 2010 Nov. 11; 363(20):1938-48.
Hudis C A, Gianni L. Triple-negative breast cancer: an unmet medical need. Oncologist. 2011; 16 Suppl 1:1-11. doi: 10.1634/theoncologist.2011-S1-01. Review. PubMed PMID: 21278435.
Lebert J M, Lester R, Powell E, Seal M, McCarthy J. Advances in the systemic treatment of triple-negative breast cancer. Curr Oncol. 2018 June; 25 (Supp11): S142-S150. Epub 2018 Jun. 13. Review. PubMed PMID: 29910657.
Luboshits G, Shina S, Kaplan O, Engelberg S, Nass D, Lifshitz-Mercer B, et al. Elevated expression of the CC chemokine regulated on activation, normal T cell expressed and secreted (RANTES) in advanced breast carcinoma. Cancer Res. 1999 Sep. 15; 59(18):4681-7. PubMed PMID: 10493525.
Jiao X, Velasco-Velazquez M A, Wang M, Li Z, Rui H, Peck A R, Korkola J E, Chen X, Xu S, DuHadaway J B, Guerrero-Rodriguez S, Addya S, Sicoli D, Mu Z, Zhang G, Stucky A, Zhang X, Cristofanilli M, Fatatis A, Gray J W, Zhong J F, Prendergast G C, Pestell R G. CCR5 Governs DNA Damage Repair and Breast Cancer Stem Cell Expansion. Cancer Res. 2018 Apr. 1; 78(7):1657-1671. doi: 10.1158/0008-5472.CAN-17-0915. Epub 2018 Jan. 22. PubMed PMID: 29358169; PubMed Central PMCID: PMC6331183.
Lanitis E, Dangaj D, Irving M, Coukos G. Mechanisms regulating T-cell infiltration and activity in solid tumors. Annals of Oncology. 2017 Sep. 21; 28 (suppl_12):xii18-32.
Malorni L, Shetty P B, De Angelis C, Hilsenbeck S, Rimawi M F, Elledge R, et al. Clinical and biologic features of triple-negative breast cancers in a large cohort of patients with long-term follow-up. Breast Cancer Res Treat. 2012 December; 136(3):795-804.
Mañes S, Mira E, Colomer R, Montero S, Real L M, Gomez-Mouton C, Jimenez-Baranda S, Garzón A, Lacalle R A, Harshman K, Ruiz A. CCR5 expression influences the progression of human breast cancer in a p53-dependent manner. Journal of Experimental Medicine. 2003 Nov. 3; 198(9):1381-9.
Moser B, Loetscher P. Lymphocyte traffic control by chemokines. Nat Immunol. 2001 February; 2(2):123-8 PubMed PMID: 11175804.
Neagu M, Constantin C, Longo C. Chemokines in the melanoma metastasis biomarkers portrait. J Immunoassay Immunochem. 2015; 36(6):559-66. PubMed PMID: 25839711.
Nielsen T O, Hsu F D, Jensen K, Cheang M, Karaca G, Hu Z, Hernandez-Boussard T, Livasy C, Cowan D, Dressler L, Akslen L A, Ragaz J, Gown A M, et al. Immunohistochemical and clinical characterization of the basal-like subtype of invasive breast carcinoma. Clin Cancer Res. 2004; 10:5367-74.
Niwa Y, Akamatsu H, Niwa H, Sumi H, Ozaki Y, Abe A. Correlation of tissue and plasma RANTES levels with disease course in patients with breast or cervical cancer. Clin Cancer Res. 2001 February; 7(2):285-9. PubMed PMID: 11234881.
Prat A, Adamo B, Cheang M C, Anders C K, Carey L A, Perou C M. Molecular characterization of basal-like and non-basal-like triple-negative breast cancer. Oncologist. 2013; 18:123-33.
Prat A, Perou C M. Deconstructing the molecular portraits of breast cancer. Mol Oncol. 2011; 5:5-23.
Sicoli D, Jiao X, Ju X, Velasco-Velazquez M, Ertel A, Addya S, Li Z, Ando S, Fatatis A, Paudyal B, Cristofanilli M, Thakur M L, Lisanti M P, Pestell R G. CCR5 receptor antagonists block metastasis to bone of v-Src oncogene-transformed metastatic prostate cancer cell lines. Cancer Res. 2014 Dec. 1; 74(23):7103-14.
Velasco-Velazquez M, Jiao X, et al. CCR5 antagonist blocks metastasis of basal breast cancer cells. Cancer Res. 2012; 72:3839-50.
Walens A, DiMarco A V, Lupo R, Kroger B R, Damrauer J S, Alvarez J V. CCL5 promotes breast cancer recurrence through macrophage recruitment in residual tumors. eLife. 2019 Apr. 16; 8:e43653.
Zetter B R, Brightman S E. Cell motility and the extracellular matrix. Curr Opin Cell Biol. 1990; 2:850-6. Review. PubMed PMID: 1964568.
Zhang Y, Yao F, Yao X, Yi C, Tan C, Wei L, et al. Role of CCL5 in invasion, proliferation and proportion of CD44+/CD24− phenotype of MCF-7 cells and correlation of CCL5 and CCR5 expression with breast cancer progression. Oncol Rep. 2009 April; 21(4):1113-21. PubMed PMID: 19288016.

1. A method of treating or preventing cancer comprising administering to a subject in need thereof an effective amount of a CCR5 binding agent.

2. The method according to claim 1, wherein the cancer comprises CCR5-positive metastatic breast cancer.

3. The method according to claim 1, wherein the cancer comprises CCR5-positive melanoma, brain cancer, glioblastoma, throat cancer, lung cancer, stomach cancer, colon cancer, colon carcinoma, breast cancer, testicular cancer, ovarian cancer, uterine cancer, pancreatic cancer, bladder cancer, esophageal cancer, appendix cancer, or prostate cancer.

4. The method according to any one of the preceding claims, wherein the CCR5 binding agent competes with CCL5 for binding to the CCR5 cell receptor.

5. The method according to any one of the preceding claims, wherein the CCR5 binding agent comprises the monoclonal antibody PA14, leronlimab, or CCR5mAb004, or a binding fragment thereof.

6. The method according to any one of the preceding claims, wherein the CCR5 binding agent is administered in combination with another cancer therapy.

7. The method of claim 1, wherein the CCR5 binding agent comprises an antibody comprising:
(a) a heavy chain variable region (VH) comprising a heavy chain complementary determining region 1 (HCDR1) of SEQ ID NO:12, a heavy chain complementary determining region 2 (HCDR2) of SEQ ID NO:13, and a heavy chain complementary determining region 3 (HCDR3) of SEQ ID NO:14; and
(b) a light chain variable region (VL) comprising a light chain complementary determining region 1 (LCDR1) of SEQ ID NO:9, a light chain complementary determining region 2 (LCDR2) of SEQ ID NO:10, and a light chain complementary determining region 3 (LCDR3) of SEQ ID NO:11.

8. The method according to any one of the preceding claims, wherein the CCR5 binding agent comprises leronlimab.

9. The method according to any one of the preceding claims, wherein the metastatic breast cancer is metastatic triple negative metastatic breast cancer.

10. The method according to any one of claims 1-8, wherein the cancer is metastatic HER2-positive breast cancer.

11. The method according to any one of the preceding claims, wherein preventing the cancer comprises slowing the growth of the cancer.

12. The method according to any one of the preceding claims, wherein preventing the cancer comprises preventing the formation of a tumor.

13. The method according to any one of the preceding claims, wherein preventing the cancer comprises preventing the formation of tumor metastases.

14. The method according to any one of the preceding claims, wherein preventing the cancer comprises limiting or reducing the size of a tumor.

15. The method according to claim 14, wherein preventing the cancer comprises limiting or reducing the size of a metastatic tumor.

16. The method according to claim 15, wherein limiting or reducing the size of a metastatic tumor comprises at least a 50% reduction in tumor volume.

17. The method according to any one of the preceding claims, wherein preventing the cancer comprises reducing the number of circulating tumor cells in the subject.

18. The method according to any one of the preceding claims, wherein preventing the cancer comprises reducing the number of epithelial mesenchymal transition cells in the subject.

19. The method according to any one of the preceding claims, wherein preventing the cancer comprises reducing the number of cancer associated macrophage-like cells in the subject.

20. The method according to any one of the preceding claims, wherein treating comprises causing the cancer progression to become stable.

21. The method according to any one of the preceding claims, further comprising administering to the subject a cellular therapy, a chemotherapeutic agent, a small molecule, or an inhibitor of CCR5/CCL5 signaling.

22. The method according to claim 21, wherein the chemotherapeutic agent comprises carboplatin.

23. The method according to claim 21, wherein the chemotherapeutic agent comprises one or more of taxotere, herceptin, and pertuzumab.

24. The method according to claim 21, wherein the inhibitor of CCR5/CCL5 signaling comprises maraviroc, vicriviroc, aplaviroc, SCH-C, TAK-779, PA14 antibody, 2D7 antibody, RoAb13 antibody, RoAb14 antibody, or 45523 antibody.

25. A method for reducing tumor burden in a subject having a CCR5+ cancer, comprising:
selecting a cancer patient suitable for treatment with a CCR5 binding agent comprising establishing that the patient has a cancer type typically known to be characterized by an elevated CCR5+ expression level or measuring a tumor biopsy from said patient for CCR5+ expression; and
administering the monoclonal antibody PA14, leronlimab, or CCR5mAb004, or a binding fragment thereof.

26. The method of claim 25, wherein the monoclonal antibody PA14, leronlimab, or CCR5mAb004, or a binding fragment thereof, is administered in weekly injections of 700 mg.

27. The method of claim 25 or claim 26, further comprising eliminating detectable brain metastasis.

28. The method of any of claims 25-27, further comprising reducing a number of brain lesions detectable by MM.

29. The method of any of claims 25-28, further comprising reducing the tumor volume of at least one brain tumor by greater than 50%.

30. The method of any of claims 25-29, further comprising, reducing the subjects CTC or EMT counts to zero.

31. A composition for treating CCR5+ cancer comprising the monoclonal antibody PA14, leronlimab, or CCR5mAb004, or a binding fragment thereof.
